(19) United States
(12) Patent Application Publication
(10) Pub. No.: US 2024/0093228 A1
(43) Pub. Date: Mar. 21, 2024

(54) COMPOSITIONS COMPRISING A NUCLEASE AND USES THEREOF

(71) Applicant: Arbor Biotechnologies, Inc., Cambridge, MA (US)

(72) Inventors: David A. SCOTT, San Francisco, CA (US); David R. CHENG, Cambridge, MA (US); Winston X. YAN, Cambridge, MA (US); Tia M. DITOMMASO, Cambridge, MA (US)

(21) Appl. No.: 18/273,349

(22) PCT Filed: Jan. 21, 2022
(86) PCT No.: PCT/US22/13375

§ 371 Date: Jul. 20, 2023

Related U.S. Application Data
(60) Provisional application No. 63/140,608, filed on Jan. 22, 2021.

(51) Int. Cl. C12N 15/85; C12N 9/22; C12N 15/11

(52) U.S. Cl. CPC C12N 15/85; C12N 9/22; C12N 15/11

(57) ABSTRACT

The present invention relates to nucleases or nucleic acids encoding the nucleases, RNA guides or nucleic acids encoding the RNA guides, processes for characterizing the nucleases and/or RNA guides, compositions comprising the nucleases and/or RNA guides, and kits and/or methods for preparing and/or using the nucleases and/or RNA guides.

### CROSS-REFERENCE TO RELATED APPLICATIONS

The present application claims priority under 35 U.S.C. § 119(e) to U.S. Provisional Application No. 63/140,608, filed Jan. 22, 2021, which application is incorporated herein by reference in its entirety.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, in one aspect, the invention provides a nuclease or a nucleic acid encoding the nuclease wherein the nuclease comprises an amino acid sequence with at least 80% identity to any one of SEQ ID NOs: 1-4.

In various aspects, the nuclease or a nucleic acid encoding the nuclease comprises a RuvC domain or a split RuvC domain. In some aspects, the nuclease or a nucleic acid encoding the nuclease comprises a catalytic residue (e.g., aspartic acid or glutamic acid).

In some aspects, the nuclease or a nucleic acid encoding the nuclease comprises one or more of the following sequences: (a) LX₁RLX₂LX₃GYEGRDDGX₄YEE (SEQ ID NO: 11), wherein X₁ is L or I, X₂ is F or I, X₃ is S or A, and X₄ is F or I; (b) TREAYAX₁QQ (SEQ ID NO: 12), wherein X₁ is E or D; (c) LX₁RYX₂ENGX₃RPR (SEQ ID NO: 13), wherein X₁ is F or L, X₂ is D or E, and X₃ is K or L; (d) KEPRX₁CWRR (SEQ ID NO: 14), wherein X₁ is F or L; (e) GX₁HX₂AFX₃RX₄WPKEVEGLD (SEQ ID NO: 15), wherein X₁ is K or D, X₂ is S or L, X₃ is T or S, and X₄ is Q or Y; (f) DPX₁NX₂HNQIKEX₃DV (SEQ ID NO: 16), wherein X₁ is G or K, X₂ is K or E, and X₃ is K or S; (g) SLKKX₁LHFGGYE (SEQ ID NO: 17), wherein X₁ is C or T; and (h) ALTRX₁AEPF (SEQ ID NO: 18), wherein X₁ is E or H.

In some aspects, the nuclease or a nucleic acid encoding the nuclease comprises an amino acid sequence with at least 95% identity to any one of SEQ ID NOs: 1-4.

In some aspects, the nuclease or a nucleic acid encoding the nuclease comprises the amino acid sequence set forth in any one of SEQ ID NOs: 1-4.

In one aspect, the invention provides an RNA guide or a nucleic acid encoding the RNA guide, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

In some aspects, the direct repeat sequence comprises the nucleotide sequence set forth in any one of SEQ ID NOs: 5-8.

In some aspects, the spacer sequence comprises about 10 to about 50 nucleotides in length. In some aspects, the spacer sequence comprises about 15 to about 35 nucleotides in length. In some aspects, the spacer sequence comprises about 20 to about 25 nucleotides in length. In some aspects, the spacer sequence comprises about 20 nucleotides.

In some aspects, the spacer sequence recognizes a target nucleic acid.

In some aspects, the target nucleic acid comprises a target sequence adjacent to a protospacer adjacent motif (PAM) sequence, wherein the PAM sequence comprises a nucleotide sequence set forth as 5′-TTN-3′ or 5′-NTTN-3′, wherein “N” is any nucleobase.

In some aspects, the PAM sequence comprises a nucleotide sequence set forth as 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′.

In some aspects, the PAM sequence comprises a nucleotide sequence set forth as 5′-GTTG-3′.

In some aspects, the invention provides a composition comprising the nuclease or the nucleic acid encoding the nuclease described herein.

In some aspects, the invention provides a composition comprising the RNA guide or the nucleic acid encoding the RNA guide described herein.

In some aspects, the invention provides a composition comprising: a) the nuclease or the nucleic acid encoding the nuclease described herein, and b) the RNA guide or the nucleic acid encoding the RNA guide described herein.

In some aspects, the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6.

In some aspects, the nuclease comprises an amino acid sequence with at least 95% identity to any one of SEQ ID NOs: 1, 2, and 4, and the RNA guide comprises SEQ ID NO: 5 or SEQ ID NO: 6.

In some aspects, the nuclease comprises any one of SEQ ID NOs: 1, 2, and 4, and the RNA guide comprises a nucleotide sequence with at least 95% sequence identity to SEQ ID NO: 5 or SEQ ID NO: 6.

In some aspects, the nuclease comprises any one of SEQ ID NOs: 1, 2, and 4, and the RNA guide comprises SEQ ID NO: 5 or SEQ ID NO: 6.

In some aspects, the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8.

In some aspects, the nuclease comprises an amino acid sequence with at least 95% identity to SEQ ID NO: 3, and the RNA guide comprises SEQ ID NO: 7 or SEQ ID NO: 8.

In some aspects, the nuclease comprises a SEQ ID NO: 3, and the RNA guide comprises a nucleotide sequence with at least 95% sequence identity to SEQ ID NO: 7 or SEQ ID NO: 8.

In some aspects, the nuclease comprises SEQ ID NO: 3, and the RNA guide comprises SEQ ID NO: 7 or SEQ ID NO: 8.

In some aspects, the composition is a delivery composition comprising a nanoparticle, a liposome, an exosome, a microvesicle, or a gene-gun.

In some aspects, the invention provides a cell comprising the nuclease or the nucleic acid encoding the nuclease described herein, the RNA guide or the nucleic acid encoding the RNA guide described herein, or the composition described herein.

In some aspects, the invention provides a method of modifying a target nucleic acid in a cell, comprising delivering to the cell:
(a) a nuclease or a nucleic acid encoding the nuclease, wherein the nuclease comprises an amino acid sequence with at least 80% identity to any one of SEQ ID NOs: 1-4; and
(b) an RNA guide or a nucleic acid encoding the RNA guide, wherein the RNA guide comprises a spacer sequence and a direct repeat sequence and the spacer sequence recognizes the target nucleic acid.

In some aspects, the nuclease comprises a RuvC domain or a split RuvC domain.

In some aspects, the nuclease comprises a catalytic residue (e.g., aspartic acid or glutamic acid).

In some aspects, the nuclease comprises one or more of the following sequences:
(a) LX₁RLX₂LX₃GYEGRDDGX₄YEE (SEQ ID NO: 11), wherein X₁ is L or I, X₂ is F or I, X₃ is S or A, and X₄ is F or I;
(b) TREAYAX₁QQ (SEQ ID NO: 12), wherein X₁ is E or D;
(c) LX₁RYX₂ENGX₃RPR (SEQ ID NO: 13), wherein X₁ is F or L, X₂ is D or E, and X₃ is K or L;
(d) KEPRX₁CWRR (SEQ ID NO: 14), wherein X₁ is F or L;
(e) GX₁HX₂AFX₃RX₄WPKEVEGLD (SEQ ID NO: 15), wherein X₁ is K or D, X₂ is S or L, X₃ is T or S, and X₄ is Q or Y;
(f) DPX₁NX₂HNQIKEX₃DV (SEQ ID NO: 16), wherein X₁ is G or K, X₂ is K or E, and X₃ is K or S;
(g) SLKKX₁LHFGGYE (SEQ ID NO: 17), wherein X₁ is C or T; and
(h) ALTRX₁AEPF (SEQ ID NO: 18), wherein X₁ is E or H.

In some aspects, the nuclease comprises an amino acid sequence with at least 95% identity to any one of SEQ ID NOs: 1-4.

In some aspects, the nuclease comprises any one of SEQ ID NOs: 1-4.

In some aspects, the RNA guide comprises a nucleotide sequence with at least 95% sequence identity to any one of SEQ ID NOs: 5-8.

In some aspects, the RNA guide comprises any one of SEQ ID NOs: 5-8.

In some aspects, the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6.

In some aspects, the nuclease comprises an amino acid sequence with at least 95% identity to any one of SEQ ID NOs: 1, 2, and 4, and the RNA guide comprises SEQ ID NO: 5 or SEQ ID NO: 6.

In some aspects, the nuclease comprises any one of SEQ ID NOs: 1, 2, and 4, and the RNA guide comprises a nucleotide sequence with at least 95% sequence identity to SEQ ID NO: 5 or SEQ ID NO: 6.

In some aspects, the nuclease comprises any one of SEQ ID NOs: 1, 2, and 4, and the RNA guide comprises SEQ ID NO: 5 or SEQ ID NO: 6.

In some aspects, the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8.

In some aspects, the nuclease comprises an amino acid sequence with at least 95% identity to SEQ ID NO: 3, and the RNA guide comprises SEQ ID NO: 7 or SEQ ID NO: 8.

In some aspects, the nuclease comprises a SEQ ID NO: 3, and RNA guide comprises a nucleotide sequence with at least 95% sequence identity to SEQ ID NO: 7 or SEQ ID NO: 8.

In some aspects, the nuclease comprises SEQ ID NO: 3, and the RNA guide comprises SEQ ID NO: 7 or SEQ ID NO: 8.

In some aspects, the RNA guide comprises a spacer sequence between 10 and 50 nucleotides in length.

In some aspects, the RNA guide comprises a spacer sequence between 15 and 25 nucleotides in length.

In some aspects, the spacer sequence comprises about 20 nucleotides in length.

In some aspects, the RNA guide recognizes the target nucleic acid comprising a target sequence adjacent to a protospacer adjacent motif (PAM) sequence, wherein the PAM sequence comprises a nucleotide sequence set forth as 5′-TTN-3′ or 5′-NTTN-3′, wherein “N” is any nucleobase.

In some aspects, the PAM sequence comprises a nucleotide sequence set forth as 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′.

In some aspects, the PAM sequence comprises a nucleotide sequence set forth as 5′-GTTG-3′.

In some aspects, the nuclease introduces a single-stranded break or a double-stranded break into the target nucleic acid.

In some aspects, the modification is an insertion, deletion, and/or substitution into the target nucleic acid.

In some aspects, the invention provides a method of binding a nuclease and an RNA guide to a target nucleic acid in a cell, the method comprising:
(a) delivering the nuclease or the nucleic acid encoding the nuclease described herein, and the RNA guide or the nucleic acid encoding the RNA guide described herein to the cell; or
(b) delivering the composition described herein to the cell.

In some aspects, the invention provides a method of introducing an insertion, deletion, or substitution into a target nucleic acid in a cell, the method comprising:
(a) delivering the nuclease or the nucleic acid encoding the nuclease described herein, and the RNA guide or the nucleic acid encoding the RNA guide described herein to the cell; or
(b) delivering the composition described herein to the cell.

In some aspects, the cell is a eukaryotic cell.

In some aspects, the cell is a prokaryotic cell.

In some aspects, the cell is a human cell.

In some aspects, the nucleic acid encoding the nuclease is operably linked to a promoter.

In some aspects, the nucleic acid encoding the nuclease is in a vector.

In some aspects, the vector comprises a retroviral vector, a lentiviral vector, a phage vector, an adenoviral vector, an adeno-associated vector, or a herpes simplex vector.

### BRIEF DESCRIPTION OF THE DRAWINGS

FIG. 1A shows an alignment of the nucleases of SEQ ID NOs: 1-4 and consensus sequence of SEQ ID NO: 19. FIG. 1B shows an alignment of the nucleases of SEQ ID NOs: 1, 2, and 4 and consensus sequence of SEQ ID NO: 20. The consensus sequences are shown at the top of the alignments.

FIG. 2 shows indels induced by the nuclease of SEQ ID NO: 4 at a VEGFA target locus (SEQ ID NO: 9) in HEK293 cells.

### DETAILED DESCRIPTION

In one aspect, the present invention provides novel nucleases and methods of use thereof. In some aspects, a composition comprising a nuclease of the present invention having one or more characteristics is described herein. In some aspects, a method of preparing a nuclease of the present invention is described. In some aspects, a method of delivering a composition comprising a nuclease of the present invention is described.

### Definitions

The present invention will be described with respect to particular embodiments and with reference to certain figures, but the invention is not limited thereto but only by the claims. Terms as set forth hereinafter are generally to be understood in their common sense unless indicated otherwise.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “catalytic residue” refers to an amino acid that activates catalysis. A catalytic residue is an amino acid that is involved (e.g., directly involved) in catalysis.

As used herein, the terms “domain” and “protein domain” refer to a distinct functional and/or structural unit of a polypeptide. In some embodiments, a domain may comprise a conserved amino acid sequence. As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain.

As used herein, the term “nuclease” refers to an enzyme capable of cleaving a phosphodiester bond. A nuclease hydrolyzes phosphodiester bonds in a nucleic acid backbone. As used herein, the term “endonuclease” refers to an enzyme capable of cleaving a phosphodiester bond between nucleotides. As used herein, “activity” and “enzymatic activity” refer to the catalytic ability of a nuclease. For example, in some embodiments, “activity” refers to the ability of a nuclease to introduce a single-stranded or double-stranded DNA break.

As used herein, the term “protospacer adjacent motif” or “PAM” refers to a DNA sequence adjacent to a “target sequence” to which a complex comprising an effector (e.g., CRISPR nuclease) and an RNA guide binds. The “target nucleic acid” is a double-stranded molecule: one strand comprises the target sequence adjacent to the PAM and is referred to as the “PAM strand” (e.g., the non-target strand or the non-spacer-complementary strand), and the other complementary strand is referred to as the “non-PAM strand” (e.g., the target strand or the spacer-complementary strand). As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM.

As used herein, the terms “reference composition,” “reference sequence,” and “reference” refer to a control, such as a negative control or a parent (e.g., a parent sequence, a parent protein, a wild-type protein, or a complex comprising a parent sequence).

As used herein, the terms “RNA guide” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a target strand (e.g., the non-PAM strand) of a target nucleic acid sequence. An RNA guide comprises a DNA targeting sequence and a direct repeat (DR) sequence. The terms CRISPR RNA (crRNA), pre-crRNA, mature crRNA, and gRNA are also used herein to refer to an RNA guide. As used herein, the term “pre-crRNA” refers to an unprocessed RNA molecule comprising a DR-spacer-DR sequence. As used herein, the term “mature crRNA” refers to a processed form of a pre-crRNA; a mature crRNA may comprise a DR-spacer sequence, wherein the DR is a truncated form of the DR of a pre-crRNA and/or the spacer is a truncated form of the spacer of a pre-crRNA.

As used herein, the term “targeting moiety” refers to a molecule or component (e.g., nucleic acid and/or RNA guide) that facilitates the targeting of another molecule or component to a target nucleic acid. In some embodiments, the targeting moiety specifically interacts or associates with the target nucleic acid.

As used herein, the term “substantially identical” refers to a sequence, polynucleotide, or polypeptide, that has a certain degree of identity to a reference sequence.

As used herein, the terms “target nucleic acid” and “target sequence” refer to a nucleic acid sequence to which a targeting moiety (e.g., RNA guide) specifically binds. In some embodiments, the DNA targeting sequence of an RNA guide binds to a target nucleic acid.

As used herein, the terms “trans-activating crRNA” and “tracrRNA” refer to an RNA molecule involved in or required for the binding of a targeting moiety (e.g., an RNA guide) to a target nucleic acid.

### Composition

In some aspects, the invention described herein comprises compositions comprising a nuclease. In some embodiments, a composition of the invention includes a nuclease, and the composition has nuclease activity. In some aspects, the invention described herein comprises compositions comprising a nuclease and a targeting moiety. In some embodiments, a composition of the invention includes a nuclease and an RNA guide sequence, and the RNA guide sequence directs the nuclease activity to a site-specific target. In some embodiments, a nuclease of the composition of the present invention is a recombinant nuclease.

In some embodiments, the composition described herein comprises an RNA-guided nuclease (e.g., a nuclease comprising multiple components). In some embodiments, a nuclease of the present invention comprises enzyme activity (e.g., a protein comprising a RuvC domain or a split RuvC domain). In some embodiments, the composition comprises a targeting moiety (e.g., an RNA guide). In some embodiments, the composition comprises a ribonucleoprotein (RNP) comprising a nuclease and a targeting moiety (e.g., RNA guide).

In some embodiments, the composition of the present invention includes a nuclease (e.g., CRISPR nuclease) described herein.

### Nuclease

In one embodiment, the nuclease is an isolated or purified nuclease.

A nucleic acid sequence encoding a nuclease described herein may be substantially identical to a reference nucleic acid sequence if the nucleic acid encoding the nuclease comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, a nuclease described herein is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

A nuclease described herein may substantially identical to a reference polypeptide if the nuclease comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

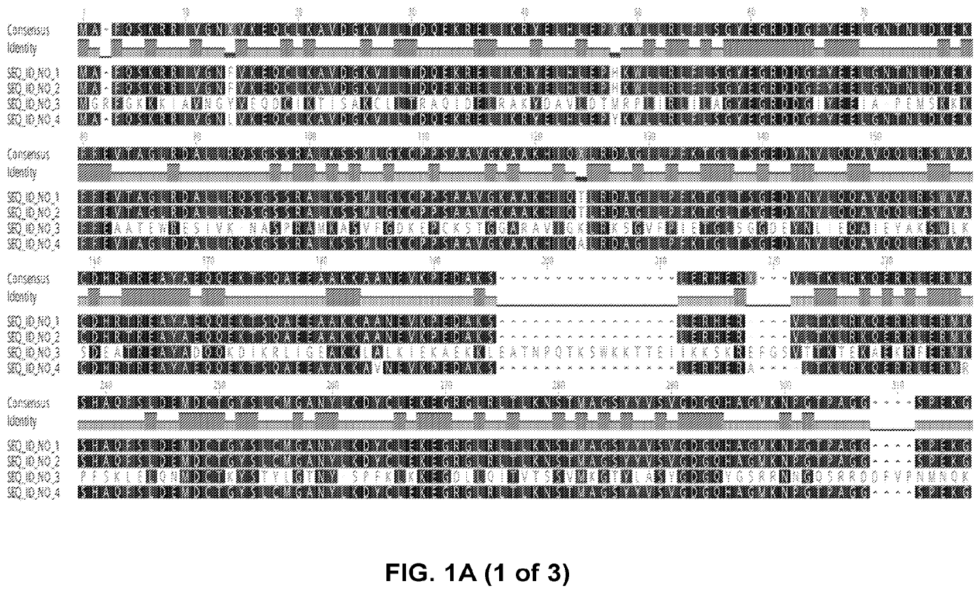

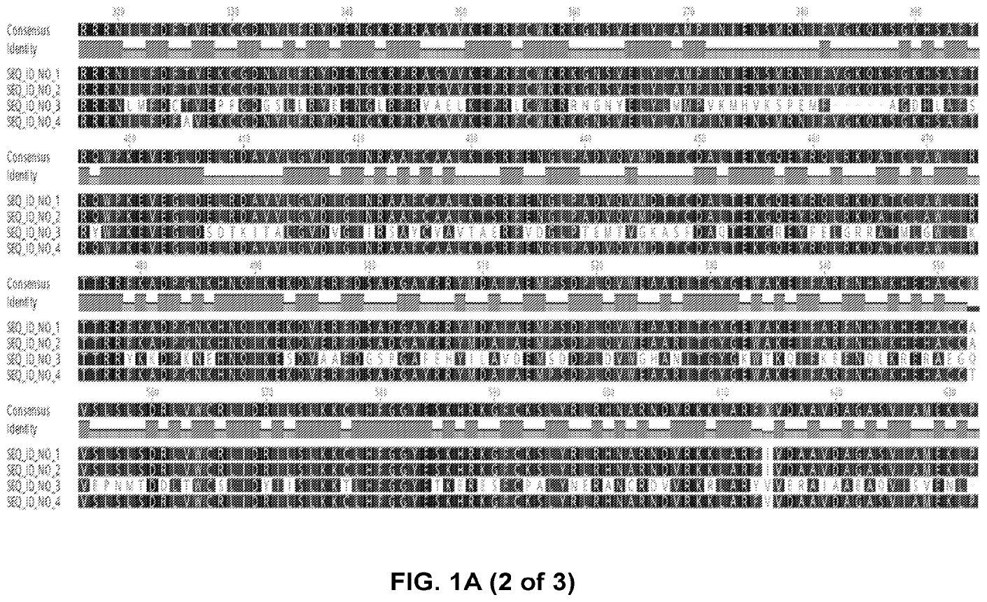

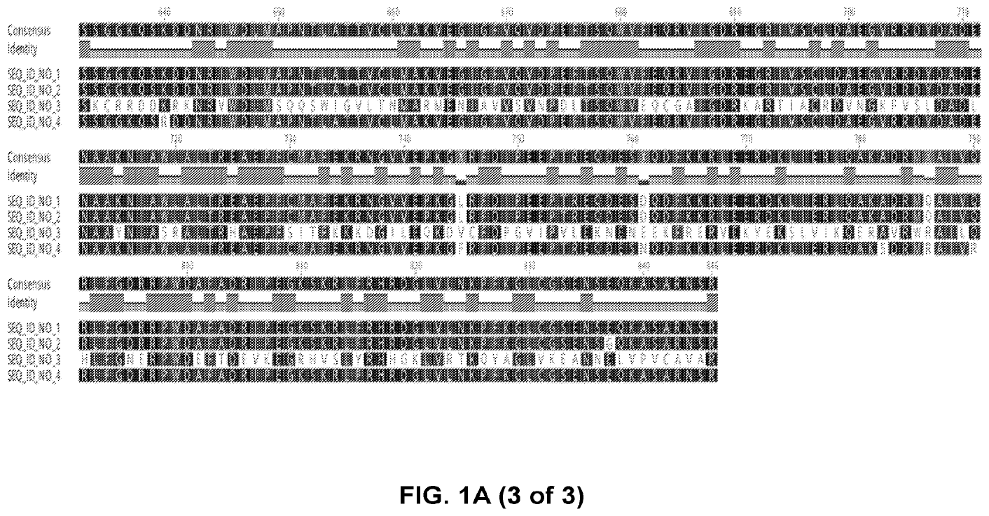

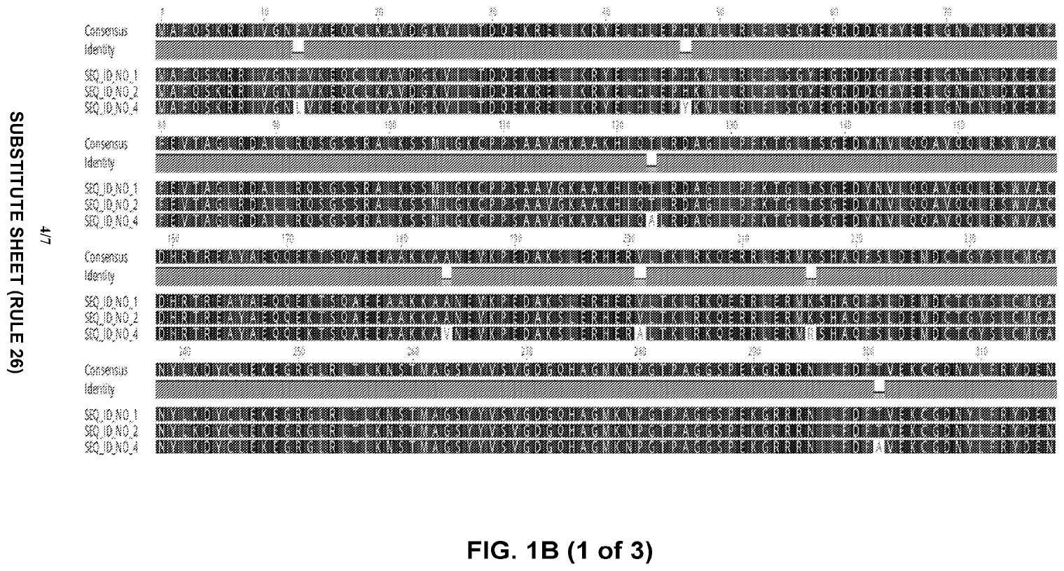

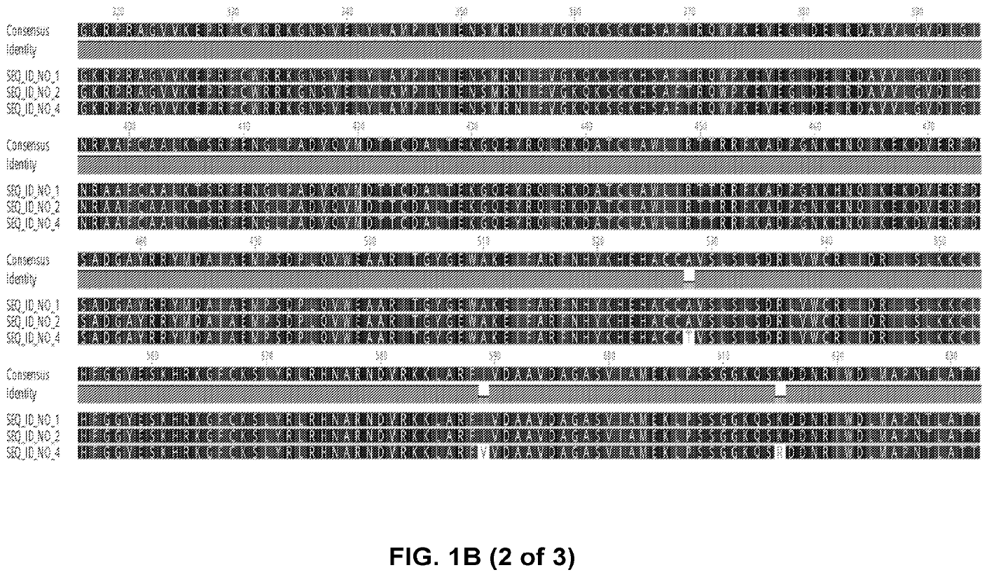

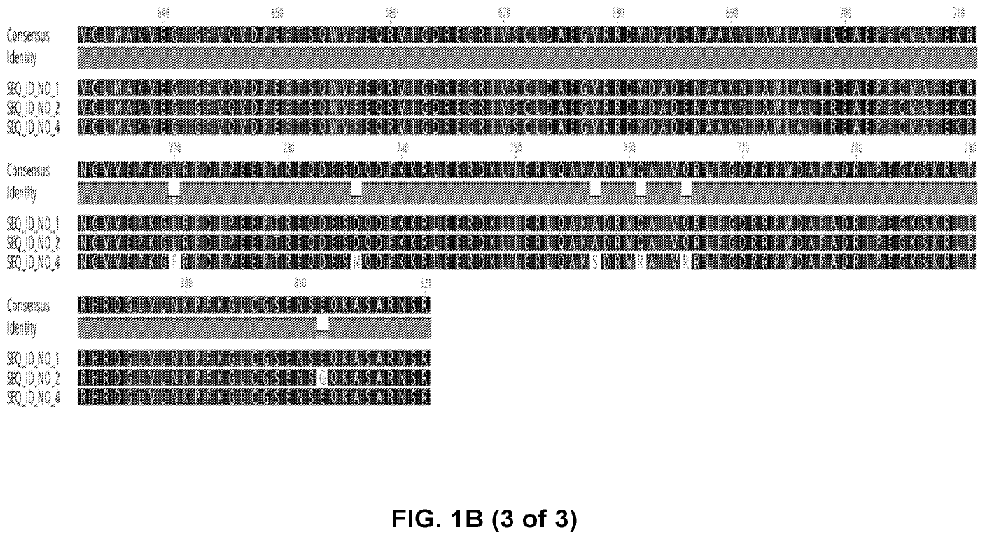

In some embodiments, a nuclease of the present invention comprises a polypeptide sequence having 50, 60, 65, 70, 75, 80, 85, 90, 91, 92, 93, 94, 95, 96, 97, 98, 99 or 100% identity to any one of SEQ ID NOs: 1-4. In some embodiments, a nuclease of the present invention comprises a polypeptide sequence having greater than 50, 60, 65, 70, 75, 80, 85, 90, 91, 92, 93, 94, 95, 96, 97, 98, 99 or 100% identity to any one of SEQ ID NOs: 1-4. The amino acid sequences corresponding to SEQ ID NOs: 1-4 are shown in Table 1. As shown in the alignment of FIG. 1A and FIG. 1B, the family of nucleases described herein comprise regions of sequence similarity.

TABLE 1
Amino acid sequences of nucleases of SEQ ID NOs: 1-4.

| SEQ | |
|---|---|
| ID NO | Amino Acid Sequence |
| 1 | MAFQSKRRIVGNFVKEQCLKAVDGKVILTDQEKRELIKRYELHLEPHKWL |
| | LRLFLSGYEGRDDGFYEELGNTNLDKEKFFEVTAGLRDALLRQSGSSRAL |
| | KSSMLGKCPPSAAVGKAAKHIQTLRDAGILPFKTGLTSGEDYNVLQQAVQ |
| | QLRSWVACDHRTREAYAEQQEKTSQAEEAAKKAANEVKPEDAKSLERHER |
| | VLTKLRKQERRLERMKSHAQFSLDEMDCTGYSLCMGANYLKDYCLEKEGR |
| | GLRLTLKNSTMAGSYYVSVGDGQHAGMKNPGTPAGGSPEKGRRRNILFDF |
| | TVEKCGDNYLFRYDENGKRPRAGVVKEPRFCWRRKGNSVELYLAMPINIE |
| | NSMRNIFVGKQKSGKHSAFTRQWPKEVEGLDELRDAVVLGVDIGINRAAF |
| | CAALKTSRFENGLPADVQVMDTTCDALTEKGQEYRQLRKDATCLAWLIRT |
| | TRRFKADPGNKHNQIKEKDVERFDSADGAYRRYMDAIAEMPSDPLQVWEA |
| | ARITGYGEWAKEIFARFNHYKHEHACCAVSLSLSDRLVWCRLIDRILSLK |
| | KCLHFGGYESKHRKGFCKSLYRLRHNARNDVRKKLARFIVDAAVDAGASV |
| | IAMEKLPSSGGKQSKDDNRIWDLMAPNTLATTVCLMAKVEGIGFVQVDPE |
| | FTSQWVFEQRVIGDREGRIVSCLDAEGVRRDYDADENAAKNIAWLALTRE |
| | AEPFCMAFEKRNGVVEPKGLRFDIPEEPTREQDESDQDFKKRLEERDKLI |
| | ERLQAKADRMQAIVQRLFGDRRPWDAFADRIPEGKSKRLFRHRDGLVLNK |
| | PFKGLCGSENSEQKASARNSR |
| 2 | MAFQSKRRIVGNFVKEQCLKAVDGKVILTDQEKRELIKRYELHLEPHKWL |
| | LRLFLSGYEGRDDGFYEELGNTNLDKEKFFEVTAGLRDALLRQSGSSRAL |
| | KSSMLGKCPPSAAVGKAAKHIQTLRDAGILPFKTGLTSGEDYNVLQQAVQ |
| | QLRSWVACDHRTREAYAEQQEKTSQAEEAAKKAANEVKPEDAKSLERHER |
| | VLTKLRKQERRLERMKSHAQFSLDEMDCTGYSLCMGANYLKDYCLEKEGR |
| | GLRLTLKNSTMAGSYYVSVGDGQHAGMKNPGTPAGGSPEKGRRRNILFDF |
| | TVEKCGDNYLFRYDENGKRPRAGVVKEPRFCWRRKGNSVELYLAMPINIE |
| | NSMRNIFVGKQKSGKHSAFTRQWPKEVEGLDELRDAVVLGVDIGINRAAF |
| | CAALKTSRFENGLPADVQVMDTTCDALTEKGQEYRQLRKDATCLAWLIRT |
| | TRRFKADPGNKHNQIKEKDVERFDSADGAYRRYMDAIAEMPSDPLQVWEA |
| | ARITGYGEWAKEIFARFNHYKHEHACCAVSLSLSDRLVWCRLIDRILSLK |
| | KCLHFGGYESKHRKGFCKSLYRLRHNARNDVRKKLARFIVDAAVDAGASV |
| | IAMEKLPSSGGKQSKDDNRIWDLMAPNTLATTVCLMAKVEGIGFVQVDPE |
| | FTSQWVFEQRVIGDREGRIVSCLDAEGVRRDYDADENAAKNIAWLALTRE |
| | AEPFCMAFEKRNGVVEPKGLRFDIPEEPTREQDESDQDFKKRLEERDKLI |
| | ERLQAKADRMQAIVQRLFGDRRPWDAFADRIPEGKSKRLFRHRDGLVLNK |
| | PFKGLCGSENSGQKASARNSR |
| 3 | MGRFGKKKIAVNGYVEQDCIKTISAKCLLTRAQIDELRAKYDAVLDTMRP |
| | LIRLILAGYEGRDDGIYEEIAPEMSKKKFFEAATEWRESIVKNASPRAMK |
| | ASVFGDKEPCKSTGGARAVIGKLRKSGVFPIETGLSGGDEYNLIEQAIEY |
| | AKSWLKSDEATREAYADQQKDIKRLIGEAKKLALKIEKAEKKLEATNPQT |
| | KSWKKTTEIIKKSKREFGSVTTKTEKAEKRFERMKPFSKLELQNMDCTKY |
| | STYLGTNYSPFKLKKEGDLLQITVTSSVMKGTYLASYGDGQYGSRRNNGQ |
| | SRRDDFVPNMNQKRRRNLMFDCTVEPFGDGSLLRYEENGLRPRVAELKEP |
| | RLCWRRRNGNYELYLMMPVKMHVKSPEMFAGDHLAFSRYWPKEVEGLDSD |
| | TKITALGVDVGIIRSAYCVAVTAERFVDGLPTEMTVGKASFDAQTEKGRE |
| | YFELGRRATMLGWLIKTTRRYKKDPKNEHNQIKESDVAAFDGSPGAFEHY |
| | ILAVDEMSDDPLDVWGHANITGYGKWTKQIFKEFNQLKRERAEGQVEPNM |
| | TDDLTWCSLIDYIISLKKTLHFGGYETKERESFCPALYNERANCRDVVRK |
| | RLARYVVERAIAAEAQVISVENLSKCRRDDKRKNRVWDLMSQQSWIGVLT |
| | NMARMENIAVVSVNPDLTSQWVEQCGAIGDRKARTIACRDVNGKFVSLDA |
| | DLNAAYNIASRALTRHAEPFSITFKKKDGILEQKDVCFDPGVIPVLEKNE |
| | NEEKFRERVEKYEKSLVIKQERAVRWRAILQHLFGNERPWDEFTDEVKEG |
| | RHVSLYRHHGKLVRTKQYAGLVKEANNELVPVCAVAR |
| 4 | MAFQSKRRIVGNLVKEQCLKAVDGKVILTDQEKRELIKRYELHLEPYKWL |
| | LRLFLSGYEGRDDGFYEELGNTNLDKEKFFEVTAGLRDALLRQSGSSRAL |
| | KSSMLGKCPPSAAVGKAAKHIQALRDAGILPFKTGLTSGEDYNVLQQAVQ |
| | QLRSWVACDHRTREAYAEQQEKTSQAEEAAKKAVNEVKPEDAKSLERHER |
| | ALTKLRKQERRLERMRSHAQFSLDEMDCTGYSLCMGANYLKDYCLEKEGR |
| | GLRLTLKNSTMAGSYYVSVGDGQHAGMKNPGTPAGGSPEKGRRRNILFDF |
| | AVEKCGDNYLFRYDENGKRPRAGVVKEPRFCWRRKGNSVELYLAMPINIE |
| | NSMRNIFVGKQKSGKHSAFTRQWPKEVEGLDELRDAVVLGVDIGINRAAF |
| | CAALKTSRFENGLPADVQVMDTTCDALTEKGQEYRQLRKDATCLAWLIRT |
| | TRRFKADPGNKHNQIKEKDVERFDSADGAYRRYMDAIAEMPSDPLQVWEA |
| | ARITGYGEWAKEIFARFNHYKHEHACCTVSLSLSDRLVWCRLIDRILSLK |
| | KCLHFGGYESKHRKGFCKSLYRLRHNARNDVRKKLARFVVDAAVDAGASV |
| | IAMEKLPSSGGKQSRDDNRIWDLMAPNTLATTVCLMAKVEGIGFVQVDPE |
| | FTSQWVFEQRVIGDREGRIVSCLDAEGVRRDYDADENAAKNIAWLALTRE |
| | AEPFCMAFEKRNGVVEPKGFRFDIPEEPTREQDESNQDFKKRLEERDKLI |
| | ERLQAKSDRMRAIVRRLFGDRRPWDAFADRIPEGKSKRLFRHRDGLVLNK |
| | PFKGLCGSENSEQKASARNSR |

In some embodiments, a nuclease of the present invention is a nuclease having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of any one of SEQ ID NOs: 1-4. Homology or identity can be determined by amino acid sequence alignment, e.g., using a program such as BLAST, ALIGN, or CLUSTAL, as described herein. In some embodiments, a nuclease having a specified degree of amino acid sequence identity to one or more reference polypeptides retains one or more characteristics, e.g., nuclease activity, as the one or more reference polypeptides.

In some embodiments, a nuclease of the present invention comprises a protein with an amino acid sequence with at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference amino acid sequence. In some embodiments, a nuclease having a specified degree of amino acid sequence identity to one or more reference polypeptides retains one or more characteristics, e.g., nuclease activity, as the reference amino acid sequence.

Also provided is a nuclease of the present invention having enzymatic activity, e.g., nuclease activity, and comprising an amino acid sequence which differs from the amino acid sequences of any one of any one of SEQ ID NOs: 1-4 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, a nuclease of the present invention comprises a RuvC domain. In some embodiments, a nuclease of the present invention comprises a split RuvC domain or two or more partial RuvC domains. For example, a nuclease comprises RuvC motifs that are not contiguous with respect to the primary amino acid sequence of the nuclease but form a RuvC domain once the protein folds. In some embodiments, the catalytic residue of a RuvC motif is a glutamic acid residue and/or an aspartic acid residue.

In some embodiments, the invention includes an isolated, recombinant, substantially pure, or non-naturally occurring nuclease comprising a RuvC domain, wherein the nuclease has enzymatic activity, e.g., nuclease activity, wherein the nuclease comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any one of SEQ ID NOs: 1-4.

In some embodiments, a nuclease described herein comprise the consensus sequence shown in FIG. 1A and FIG. 1B.

FIG. 1A Consensus Sequence
(SEQ ID NO: 19)
MA-FQSKRRIVGNXVKEQCLKAVDGKVILTDQEKRELIKRYELHLEPXK
WLLRLFLSGYEGRDDGFYEELGNTNLDKEKFFEVTAGLRDALLRQSGSS
RALKSSMLGKCPPSAAVGKAAKHIQXLRDAGILPFKTGLTSGEDYNVLQ
QAVQQLRSWVACDHRTREAYAEQQEKTSQAEEAAKKAANEVKPEDAKS-
---------------LERHERX---VLTKLRKQERRLERMKSHAQFSLD
EMDCTGYSLCMGANYLKDYCLEKEGRGLRLTLKNSTMAGSYYVSVGDGQ
HAGMKNPGTPAGG----SPEKGRRRNILFDFTVEKCGDNYLFRYDENGK
RPRAGVVKEPRFCWRRKGNSVELYLAMPINIENSMRNIFVGKQKSGKHS
AFTRQWPKEVEGLDELRDAVVLGVDIGINRAAFCAALKTSRFENGLPAD
VQVMDTTCDALTEKGQEYRQLRKDATCLAWLIRTTRRFKADPGNKHNQI
KEKDVERFDSADGAYRRYMDAIAEMPSDPLQVWEAARITGYGEWAKEIF
ARFNHYKHEHACCXVSLSLSDRLVWCRLIDRILSLKKCLHFGGYESKHR
KGFCKSLYRLRHNARNDVRKKLARFXVDAAVDAGASVIAMEKLPSSGGK
QSKDDNRIWDLMAPNTLATTVCLMAKVEGIGFVQVDPEFTSQWVFEQRV
IGDREGRIVSCLDAEGVRRDYDADENAAKNIAWLALTREAEPFCMAFEK
RNGVVEPKGXRFDIPEEPTREQDESXQDFKKRLEERDKLIERLQAKADR
MXAIVQRLFGDRRPWDAFADRIPEGKSKRLFRHRDGLVLNKPFKGLCGS
ENSEQKASARNSR
FIG. 1B Consensus Sequence
(SEQ ID NO: 20)
MAFQSKRRIVGNFVKEQCLKAVDGKVILTDQEKRELIKRYELHLEPHKW
LLRLFLSGYEGRDDGFYEELGNTNLDKEKFFEVTAGLRDALLRQSGSSR
ALKSSMLGKCPPSAAVGKAAKHIQTLRDAGILPFKTGLTSGEDYNVLQQ
AVQQLRSWVACDHRTREAYAEQQEKTSQAEEAAKKAANEVKPEDAKSLE
RHERVLTKLRKQERRLERMKSHAQFSLDEMDCTGYSLCMGANYLKDYCL
EKEGRGLRLTLKNSTMAGSYYVSVGDGQHAGMKNPGTPAGGSPEKGRRR
NILFDFTVEKCGDNYLFRYDENGKRPRAGVVKEPRFCWRRKGNSVELYL
AMPINIENSMRNIFVGKQKSGKHSAFTRQWPKEVEGLDELRDAVVLGVD
IGINRAAFCAALKTSRFENGLPADVQVMDTTCDALTEKGQEYROLRKDA
TCLAWLIRTTRRFKADPGNKHNQIKEKDVERFDSADGAYRRYMDAIAEM
PSDPLQVWEAARITGYGEWAKEIFARFNHYKHEHACCAVSLSLSDRLVW
CRLIDRILSLKKCLHFGGYESKHRKGFCKSLYRLRHNARNDVRKKLARF
IVDAAVDAGASVIAMEKLPSSGGKQSKDDNRIWDLMAPNTLATTVCLMA
KVEGIGFVQVDPEFTSQWVFEQRVIGDREGRIVSCLDAEGVRRDYDADE
NAAKNIAWLALTREAEPFCMAFEKRNGVVEPKGLRFDIPEEPTREQDES
DQDFKKRLEERDKLIERLQAKADRMQAIVQRLFGDRRPWDAFADRIPEG
KSKRLFRHRDGLVLNKPFKGLCGSENSEQKASARNSR

In some embodiments, a nuclease described herein comprises a portion of the consensus sequence shown in FIG. 1A and FIG. 1B, e.g. a conserved sequence of FIG. 1A and FIG. 1B. For example, in some embodiments, a nuclease comprises a sequence set forth as LX₁RLX₂LX₃GYEGRDDGX₄YEE (SEQ ID NO: 11), wherein X₁ is L or I, X₂ is F or I, X₃ is S or A, and X₄ is F or I. In some embodiments, a nuclease comprises a sequence set forth as TREAYAX₁QQ (SEQ ID NO: 12), wherein X₁ is E or D. In some embodiments, a nuclease comprises a sequence set forth as LX₁RYX₂ENGX₃RPR (SEQ ID NO: 13), wherein X₁ is F or L, X₂ is D or E, and X₃ is K or L. In some embodiments, a nuclease comprises a sequence set forth as KEPRX₁CWRR (SEQ ID NO: 14), wherein X₁ is F or L. In some embodiments, a nuclease comprises a sequence set forth as GX₁HX₂AFX₃RX₄WPKEVEGLD (SEQ ID NO: 15), wherein X₁ is K or D, X₂ is S or L, X₃ is T or S, and X₄ is Q or Y. In some embodiments, a nuclease comprises a sequence set forth as DPX₁NX₂HNQIKEX₃DV (SEQ ID NO: 16), wherein X₁ is G or K, X₂ is K or E, and X₃ is K or S. In some embodiments, a nuclease comprises a sequence set forth as SLKKX₁LHFGGYE (SEQ ID NO: 17), wherein X₁ is C or T. In some embodiments, a nuclease comprises a sequence set forth as ALTRX₁AEPF (SEQ ID NO: 18), wherein X₁ is E or H.

### Biochemical Characteristics

In some embodiments, the biochemistry of a nuclease described herein is analyzed using one or more assays. In some embodiments, the biochemical characteristics of a nuclease of the present invention are analyzed in mammalian cells, as described in Example 3.

Described herein are compositions and methods relating to a nuclease of the present invention. The compositions and methods are based, in part, on the observation that cloned and expressed polypeptides of the present invention have CRISPR nuclease activity.

In some embodiments, a nuclease and an RNA guide as described herein form a complex (e.g., an RNP). In some embodiments, the complex includes other components. In some embodiments, the complex is activated upon binding to a nucleic acid substrate that has complementarity to a spacer sequence in the RNA guide (e.g., a spacer-complementary strand of a target nucleic acid). In some embodiments, the target nucleic acid is a double-stranded DNA (dsDNA). In some embodiments, the sequence-specificity requires a complete match of the spacer sequence in the RNA guide to the non-PAM strand of a target nucleic acid. In other embodiments, the sequence specificity requires a partial (contiguous or non-contiguous) match of the spacer sequence in the RNA guide to the non-PAM strand of a target nucleic acid.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In one embodiment, the target nucleic acid is an extrachromosomal nucleic acid. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA. In some embodiments, the target nucleic acid is present in a cell-free environment. In some embodiments, the target nucleic acid is an isolated vector, such as a plasmid. In some embodiments, the target nucleic acid is an ultrapure plasmid.

In some embodiments, the complex becomes activated upon binding to the target substrate. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the complex reverts to an inactive state.

In some embodiments, the RNA guide or a complex comprising the RNA guide and a nuclease described herein binds to a target nucleic acid at a sequence defined by the region of complementarity between the RNA guide and the target nucleic acid. In some embodiments, the PAM sequence described herein is located directly upstream of the target sequence of the target nucleic acid (e.g., directly 5′ of the target sequence). In some embodiments, the PAM sequence described herein is located directly 5′ of the target sequence on the non-spacer-complementary strand (e.g., non-target strand) of the target nucleic acid.

In some embodiments, a nuclease of the present invention targets a sequence adjacent to a PAM, wherein the PAM comprises a nucleotide sequence set forth as 5′-TTN-3′ or 5′-NTTN-3′, wherein “N” is any nucleobase. For example, in some embodiments, a nuclease described herein recognizes a PAM sequence of 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′. In some embodiments, a nuclease described herein recognizes a PAM sequence of 5′-GTTG-3′.

In some embodiments, a nuclease described herein cleaves dsDNA. In some embodiments, a nuclease described herein is a nickase (e.g., the nuclease cleaves one strand of a double-stranded target nucleic acid).

In some embodiments, a nuclease of the present invention has enzymatic activity, e.g., nuclease activity, over a broad range of pH conditions. In some embodiments, the nuclease has enzymatic activity, e.g., nuclease activity, at a pH of from about 3.0 to about 12.0. In some embodiments, the nuclease has enzymatic activity at a pH of from about 4.0 to about 10.5. In some embodiments, the nuclease has enzymatic activity at a pH of from about 5.5 to about 8.5. In some embodiments, the nuclease has enzymatic activity at a pH of from about 6.0 to about 8.0. In some embodiments, the nuclease has enzymatic activity at a pH of about 7.0.

In some embodiments, a nuclease of the present invention has enzymatic activity, e.g., nuclease activity, at a temperature range of from about 10° C. to about 100° C. In some embodiments, a nuclease of the present invention has enzymatic activity at a temperature range from about 20° C. to about 90° C. In some embodiments, a nuclease of the present invention has enzymatic activity at a temperature of about 20° C. to about 25° C. or at a temperature of about 37° C.

In some embodiments wherein a nuclease of the present invention induces double-stranded breaks or single-stranded breaks in a target nucleic acid (e.g., genomic DNA), the double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, binding of a nuclease/RNA guide complex to a target locus in a cell recruits one or more endogenous cellular molecules or pathways other than DNA repair pathways to modify the target nucleic acid. In some embodiments, binding of a nuclease/RNA guide complex blocks access of one or more endogenous cellular molecules or pathways to the target nucleic acid, thereby modifying the target nucleic acid. For example, binding of a nuclease/RNA guide complex may block endogenous transcription or translation machinery to decrease the expression of the target nucleic acid.

Variants

In some embodiments, the present invention includes variants of a nuclease described herein. In some embodiments, a nuclease described herein can be mutated at one or more amino acid residues to modify one or more functional activities. For example, in some embodiments, a nuclease of the present invention is mutated at one or more amino acid residues to modify its nuclease activity (e.g., cleavage activity). For example, in some embodiments, a nuclease may comprise one or more mutations that increase the ability of the nuclease to cleave a target nucleic acid. In some embodiments, a nuclease is mutated at one or more amino acid residues to modify its ability to functionally associate with an RNA guide. In some embodiments, a nuclease is mutated at one or more amino acid residues to modify its ability to functionally associate with a target nucleic acid.

In some embodiments, a variant nuclease has a conservative or non-conservative amino acid substitution, deletion or addition. In some embodiments, the variant nuclease has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Leu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a nuclease disclosed herein are mutated to an Arg residue. In some embodiments, one or more residues of a nuclease disclosed herein are mutated to a Gly residue.

A variety of methods are known in the art that are suitable for generating modified polynucleotides that encode variant nucleases of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

In some embodiments, a nuclease of the present invention comprises an alteration at one or more (e.g., several) amino acids in the nuclease, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more.

As used herein, a “biologically active portion” is a portion that maintains the function (e.g. completely, partially, minimally) of a nuclease (e.g., a “minimal” or “core” domain). In some embodiments, a nuclease fusion protein is useful in the methods described herein. Accordingly, in some embodiments, a nucleic acid encoding the fusion nuclease is described herein. In some embodiments, all or a portion of one or more components of the nuclease fusion protein are encoded in a single nucleic acid sequence.

Although the changes described herein may be one or more amino acid changes, changes to a nuclease may also be of a substantive nature, such as fusion of polypeptides as amino- and/or carboxyl-terminal extensions. For example, nuclease may contain additional peptides, e.g., one or more peptides. Examples of additional peptides may include epitope peptides for labelling, such as a polyhistidine tag (His-tag), Myc, and FLAG. In some embodiments, a nuclease described herein can be fused to a detectable moiety such as a fluorescent protein (e.g., green fluorescent protein (GFP) or yellow fluorescent protein (YFP)).

A nuclease described herein can be modified to have diminished nuclease activity, e.g., nuclease inactivation of at least 50%, at least 60%, at least 70%, at least 80%, at least 90%, at least 95%, at least 97%, or 100%, as compared to a reference nuclease. Nuclease activity can be diminished by several methods known in the art, e.g., introducing mutations into the RuvC domain (e.g., one or more catalytic residues of the RuvC domain).

In some embodiments, the nuclease described herein can be self-inactivating. See, Epstein et al., “Engineering a Self-Inactivating CRISPR System for AAV Vectors,” Mol. Ther., 24 (2016): S50, which is incorporated by reference in its entirety.

Nucleic acid molecules encoding the nucleases described herein can further be codon-optimized. The nucleic acid can be codon-optimized for use in a particular host cell, such as a bacterial cell or a mammalian cell.

### Targeting Moiety

In some embodiments, the composition described herein comprises a targeting moiety.

The targeting moiety may be substantially identical to a reference nucleic acid sequence if the targeting moiety comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the targeting moiety has at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence.

RNA Guide Sequence

In some embodiments, the targeting moiety comprises, or is, an RNA guide sequence. In some embodiments, the RNA guide sequence directs a nuclease described herein to a particular nucleic acid sequence. Those skilled in the art reading the below examples of particular kinds of RNA guide sequences will understand that, in some embodiments, an RNA guide sequence is site-specific. That is, in some embodiments, an RNA guide sequence associates specifically with one or more target nucleic acid sequences (e.g., specific DNA or genomic DNA sequences) and not to non-targeted nucleic acid sequences (e.g., non-specific DNA or random sequences).

In some embodiments, the composition as described herein comprises an RNA guide sequence that associates with a nuclease described herein and directs a nuclease to a target nucleic acid sequence (e.g., DNA). The RNA guide sequence may associate with a nucleic acid sequence and alter functionality of a nuclease (e.g., alters affinity of the nuclease to a molecule, e.g., at least 10%, 15%, 20%, 25%, 30%, 35%, 40%, 45%, 50%, 55%, 60%, 65%, 70%, 75%, 80%, 85%, 90%, 95%, or more).

The RNA guide sequence may target (e.g., associate with, be directed to, contact, or bind) one or more nucleotides of a sequence, e.g., a site-specific sequence or a site-specific target. In some embodiments, a nuclease (e.g., a nuclease plus an RNA guide) is activated upon binding to a nucleic acid substrate.

In some embodiments, an RNA guide sequence comprises a spacer sequence. In some embodiments, the spacer sequence of the RNA guide sequence may be generally designed to have a length of between 15 and 50 nucleotides and be complementary to a specific nucleic acid sequence (e.g., the target strand of a target nucleic acid). In some embodiments, the spacer is about 15-25 in length, about 15-20 nucleotides in length, about 20-25 nucleotides in length, about 25-30 nucleotides in length, about 30-35 nucleotides in length, about 35-40 nucleotides in length, about 40-45 nucleotides in length, or about 45-50 nucleotides in length). In some embodiments, the spacer is 20 nucleotides in length. In some particular embodiments, the RNA guide sequence may be designed to be complementary to a specific DNA strand of a genomic locus (e.g., a target strand). In some embodiments, the spacer sequence is designed to be complementary to a specific DNA strand of a genomic locus (e.g., a target strand).

In certain embodiments, the RNA guide sequence includes, consists essentially of, or comprises a direct repeat sequence linked to a sequence or spacer sequence. In some embodiments, the RNA guide sequence includes a direct repeat sequence and a spacer sequence or a direct repeat-spacer-direct repeat sequence. In some embodiments, the RNA guide sequence includes a truncated direct repeat sequence and a spacer sequence, which is typical of processed or mature crRNA. In some embodiments, a nuclease forms a complex with the RNA guide sequence, and the RNA guide sequence directs the complex to associate with site-specific target nucleic acid that is complementary to at least a portion of the RNA guide sequence. For example, in certain embodiments, the RNA guide sequence directs the complex to associate with the target strand of a target nucleic acid, wherein the target strand is complementary to at least a portion (e.g., the spacer) of the RNA guide sequence.

In some embodiments, the RNA guide sequence comprises a sequence, e.g., spacer sequence, at least 80%, at least 90%, at least 95%, at least 96%, at least 97%, at least 98%, at least 99% complementary to the target strand of a target nucleic acid. In some embodiments, the RNA guide spacer sequence comprises a sequence at least 80%, at least 90%, at least 95%, at least 96%, at least 97%, at least 98%, at least 99% complementary to a DNA sequence. In some embodiments, the RNA guide spacer sequence comprises a sequence at least 80%, at least 90%, at least 95%, at least 96%, at least 97%, at least 98%, at least 99% complementary to the target strand of a target nucleic acid sequence. In some embodiments, the RNA guide spacer sequence comprises a sequence at least 80%, at least 90%, at least 95%, at least 96%, at least 97%, at least 98%, at least 99% complementary to a genomic sequence.

In some embodiments, the target sequence of the present invention is adjacent to a PAM comprising a nucleotide sequence set forth as 5′-TTN-3′ or 5′-NTTN-3′, wherein “N” is any nucleobase. For example, in some embodiments, the target sequence of the present invention is adjacent to a PAM comprising a nucleotide sequence set forth as to a 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′. In some embodiments, the target sequence of the present invention is adjacent to a PAM comprising a 5′-GTTG-3′ sequence.

In some embodiments, an RNA guide (e.g., the spacer of the RNA guide) of the present invention binds a target nucleic acid comprising a target sequence, wherein the target sequence is adjacent to a PAM, wherein the PAM comprises a nucleotide sequence set forth as 5′-TTN-3′ or 5′-NTTN-3′, wherein “N” is any nucleobase. For example, in some embodiments, an RNA guide (e.g., the spacer of the RNA guide) described herein binds to a target nucleic acid comprising a target sequence, wherein the target sequence is adjacent to a 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′ sequence. In some embodiments, an RNA guide (e.g., the spacer of the RNA guide) described herein binds to a target nucleic acid comprising a target sequence, wherein the target sequence is adjacent to a 5′-GTTG-3′ sequence.

In some embodiments, a nuclease described herein includes one or more (e.g., two, three, four, five, six, seven, eight, or more) RNA guide sequences, e.g., RNA guides.

In some embodiments, the RNA guide has an architecture similar to, for example International Publication Nos. WO 2014/093622 and WO 2015/070083, the entire contents of each of which are incorporated herein by reference.

In some embodiments, an RNA guide sequence of the present invention comprises a direct repeat sequence having 80, 85, 90, 91, 92, 93, 94, 95, 96, 97, 98, 99 or 100% identity the direct repeat sequences of Table 2. In some embodiments, an RNA guide of the present invention comprises a direct repeat sequence having greater than 80, 85, 90, 91, 92, 93, 94, 95, 96, 97, 98, 99 or 100% identity to the direct repeat sequences of Table 2.

TABLE 2
Direct repeat sequences.

| Nuclease | |
|---|---|
| SEQ ID NO | Direct Repeat Sequence |
| 1 | GUGUAGGCCUCCUCUGAAUGGGGUGGCUAAUGACAC |
| | (SEQ ID NO: 5) |
| | GUGUCAUUAGCCACCCCAUUCAGAGGAGGCCUACAC |
| | (SEQ ID NO: 6) |
| 2 | GUGUAGGCCUCCUCUGAAUGGGGUGGCUAAUGACAC |
| | (SEQ ID NO: 5) |
| | GUGUCAUUAGCCACCCCAUUCAGAGGAGGCCUACAC |
| | (SEQ ID NO: 6) |
| 3 | CAUUCAGAACGGAUCAACAC (SEQ ID NO: 7) |
| | GUGUUGAUCCGUUCUGAAUG (SEQ ID NO: 8) |
| 4 | GUGUAGGCCUCCUCUGAAUGGGGUGGCUAAUGACAC |
| | (SEQ ID NO: 5) |
| | GUGUCAUUAGCCACCCCAUUCAGAGGAGGCCUACAC |
| | (SEQ ID NO: 6) |

In some embodiments, a CRISPR-associated protein and an RNA guide (e.g., an RNA guide comprising a direct repeat and a spacer) form a complex. In some embodiments, a CRISPR-associated protein and an RNA guide (e.g., an RNA guide comprising direct repeat-spacer-direct repeat sequence or pre-crRNA) form a complex. In some embodiments, the complex binds a target nucleic acid.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5 or SEQ ID NO: 6. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5 or SEQ ID NO: 6. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 7 or SEQ ID NO: 8. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 7.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5 or SEQ ID NO: 6. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 5.

Unless otherwise noted, all compositions and nucleases provided herein are made in reference to the active level of that composition or nuclease, and are exclusive of impurities, for example, residual solvents or by-products, which may be present in commercially available sources. Nuclease component weights are based on total active protein. All percentages and ratios are calculated by weight unless otherwise indicated. All percentages and ratios are calculated based on the total composition unless otherwise indicated. In the exemplified composition, the nuclease levels are expressed by pure enzyme by weight of the total composition and unless otherwise specified, the ingredients are expressed by weight of the total compositions.

Modifications

The RNA guide sequence or any of the nucleic acid sequences encoding a nuclease may include one or more covalent modifications with respect to a reference sequence, in particular the parent polyribonucleotide, which are included within the scope of this invention.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide sequence or any of the nucleic acid sequences encoding components of a nuclease may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Vectors

The present invention provides a vector for expressing a nuclease described herein or nucleic acids encoding a nuclease described herein may be incorporated into a vector. In some embodiments, a vector of the invention includes a nucleotide sequence encoding a nuclease described herein. In some embodiments, a vector of the invention includes a nucleotide sequence encoding a nuclease described herein.

The present invention also provides a vector that may be used for preparation of a nuclease described herein or compositions comprising a nuclease described herein. In some embodiments, the invention includes the composition or vector described herein in a cell. In some embodiments, the invention includes a method of expressing the composition comprising a nuclease of the present invention, or vector or nucleic acid encoding the nuclease, in a cell. The method may comprise the steps of providing the composition, e.g., vector or nucleic acid, and delivering the composition to the cell.

Expression of natural or synthetic polynucleotides is typically achieved by operably linking a polynucleotide encoding the gene of interest, e.g., nucleotide sequence encoding a nuclease of the present invention, to a promoter and incorporating the construct into an expression vector. The expression vector is not particularly limited as long as it includes a polynucleotide encoding a nuclease of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of a nuclease of the present invention from a polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding a nuclease of the present invention has been transferred into the host cells and then expressed without fail.

The preparation method for recombinant expression vectors is not particularly limited, and examples thereof include methods using a plasmid, a phage or a cosmid.

### Cells

The nucleases described herein can be introduced into a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as nuclease polypeptide encoding vector and RNA guide) is used to establish a new cell line comprising one or more vector-derived sequences to establish a new cell line comprising modification to the target nucleic acid or target locus. In some embodiments, the cell is an immortal or immortalized cell.

In some embodiments, the method comprises introducing into a host cell one or more nucleic acids comprising nucleotide sequences encoding a DNA-targeting RNA (e.g., RNA guide) and/or the nuclease. In one embodiment, a cell comprising a target DNA is in vitro, in vivo, or ex vivo. In other embodiments, nucleic acids comprising nucleotide sequences encoding a DNA-targeting RNA (e.g., RNA guide) and/or the nuclease include recombinant expression vectors e.g., including but not limited to adeno-associated virus constructs, recombinant adenoviral constructs, recombinant lentiviral constructs, recombinant retroviral constructs, and the like.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model.

In some embodiments, a method for modifying a target DNA molecule in a cell is provided. The method comprises contacting the target DNA molecule inside of a cell with a nuclease described herein; and a single molecule DNA-targeting RNA comprising, in 5′ to 3′ order, a first nucleotide segment that hybridizes with a target sequence of the target DNA molecule; a nucleotide linker; and a second nucleotide segment that hybridizes with the first nucleotide segment to form a double-stranded RNA duplex. The variant polypeptide forms a complex with the single molecule DNA-targeting RNA inside the cell and the target DNA molecule is modified.

### Preparation

In some embodiments, a nuclease of the present invention can be prepared by (I) culturing bacteria which produce a nuclease of the present invention, isolating the nuclease, and optionally, purifying the nuclease. The nuclease can be also prepared by (II) a known genetic engineering technique, specifically, by isolating a gene encoding a nuclease of the present invention from bacteria, constructing a recombinant expression vector, and then transferring the vector into an appropriate host cell for expression of a recombinant protein. Alternatively, a nuclease can be prepared by (III) an in vitro coupled transcription-translation system. Bacteria that can be used for preparation of a nuclease of the present invention are not particularly limited as long as they can produce a nuclease of the present invention. Some non-limiting examples of the bacteria include E. coli cells described herein.

Methods of Expression

The present invention includes a method for protein expression, comprising translating a nuclease described herein.

In some embodiments, a host cell described herein is used to express a nuclease. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of a nuclease. After expression of the nuclease, the host cells can be collected and nuclease purified from the cultures etc. according to conventional methods (for example, filtration, centrifugation, cell disruption, gel filtration chromatography, ion exchange chromatography, etc.).

In some embodiments, the methods for nuclease expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of a nuclease. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of a nuclease.

A variety of methods can be used to determine the level of production of a mature nuclease in a host cell. Such methods include, but are not limited to, for example, methods that utilize either polyclonal or monoclonal antibodies specific for a nuclease. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of a nuclease in a cell, comprising providing a polyribonucleotide encoding the nuclease to a host cell wherein the polyribonucleotide encodes the nuclease, expressing the nuclease in the cell, and obtaining the nuclease from the cell.

### Delivery

Nucleases, RNA guides, and/or compositions described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding a nuclease, RNA guide, donor DNA, etc.), one or more transcripts thereof, and/or a pre-formed nuclease/RNA guide complex to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells.

All references and publications cited herein are hereby incorporated by reference.

### Enumerated Embodiments

The following enumerated embodiments are provided, the numbering of which is not to be construed as designating levels of importance:

Embodiment 1 provides a nuclease or a nucleic acid encoding the nuclease wherein the nuclease comprises an amino acid sequence with at least 80% identity to any one of SEQ ID NOs: 1-4.

Embodiment 2 provides the nuclease or the nucleic acid encoding the nuclease embodiment 1, wherein the nuclease comprises a RuvC domain or a split RuvC domain.

Embodiment 3 provides the nuclease or the nucleic acid encoding the nuclease of embodiment 1 or 2, wherein the nuclease comprises a catalytic residue (e.g., aspartic acid or glutamic acid).

Embodiment 4 provides the nuclease or the nucleic acid encoding the nuclease of any previous embodiment, wherein the nuclease comprises one or more of the following sequences:
(a) LX₁RLX₂LX₃GYEGRDDGX₄YEE (SEQ ID NO: 11), wherein X₁ is L or I, X₂ is F or I, X₃ is S or A, and X₄ is F or I;
(b) TREAYAX₁QQ (SEQ ID NO: 12), wherein X₁ is E or D;
(c) LX₁RYX₂ENGX₃RPR (SEQ ID NO: 13), wherein X₁ is F or L, X₂ is D or E, and X₃ is K or L;
(d) KEPRX₁CWRR (SEQ ID NO: 14), wherein X₁ is F or L;
(e) GX₁HX₂AFX₃RX₄WPKEVEGLD (SEQ ID NO: 15), wherein X₁ is K or D, X₂ is S or L, X₃ is T or S, and X₄ is Q or Y;
(f) DPX₁NX₂HNQIKEX₃DV (SEQ ID NO: 16), wherein X₁ is G or K, X₂ is K or E, and X₃ is K or S;
(g) SLKKX₁LHFGGYE (SEQ ID NO: 17), wherein X₁ is C or T; and
(h) ALTRX₁AEPF (SEQ ID NO: 18), wherein X₁ is E or H.

Embodiment 5 provides the nuclease or the nucleic acid encoding the nuclease of any previous embodiment, wherein the nuclease comprises an amino acid sequence with at least 95% identity to any one of SEQ ID NOs: 1-4.

Embodiment 6 provides the nuclease or the nucleic acid encoding the nuclease of any previous embodiment, wherein the nuclease comprises any one of SEQ ID NOs: 1-4.

Embodiment 7 provides an RNA guide or a nucleic acid encoding the RNA guide, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

Embodiment 8 provides the RNA guide or the nucleic acid encoding the RNA guide of embodiment 7, wherein the direct repeat sequence comprises any one of SEQ ID NOs: 5-8.

Embodiment 9 provides the RNA guide or the nucleic acid encoding the RNA guide of embodiment 7 or 8, wherein the spacer sequence comprises about 10 to about 50 nucleotides in length.

Embodiment 10 provides the RNA guide or the nucleic acid encoding the RNA guide of any of embodiments 7-9, wherein the spacer sequence comprises about 15 to about 35 nucleotides in length.

Embodiment 11 provides the RNA guide or the nucleic acid encoding the RNA guide of any of embodiments 7-10, wherein the spacer sequence comprises about 20 to about 25 nucleotides in length.

Embodiment 12 provides the RNA guide or the nucleic acid encoding the RNA guide of any of embodiments 7-11, wherein the spacer sequence recognizes a target nucleic acid.

Embodiment 13 provides the RNA guide or the nucleic acid encoding the RNA guide of embodiment 12, wherein the target nucleic acid comprises a target sequence adjacent to a protospacer adjacent motif (PAM) sequence, wherein the PAM sequence comprises a nucleotide sequence set forth as 5′-TTN-3′ or 5′-NTTN-3′, wherein “N” is any nucleobase.

Embodiment 14 provides the RNA guide or the nucleic acid encoding the RNA guide of embodiment 13, wherein the PAM sequence comprises a nucleotide sequence set forth as 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′.

Embodiment 15 provides the RNA guide or the nucleic acid encoding the RNA guide of embodiment 14, wherein the PAM sequence comprises a nucleotide sequence set forth as 5′-GTTG-3′.

Embodiment 16 provides a composition comprising the nuclease or the nucleic acid encoding the nuclease of any one of embodiments 1-6.

Embodiment 17 provides a composition comprising the RNA guide or the nucleic acid encoding the RNA guide of any one of embodiments 7-15.

Embodiment 18 provides a composition comprising: a) the nuclease or the nucleic acid encoding the nuclease of any one of embodiments 1-6, and b) the RNA guide or the nucleic acid encoding the RNA guide of any one of embodiments 7-15.

Embodiment 19 provides the composition of embodiment 18, wherein:
(a) the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6;
(b) the nuclease comprises an amino acid sequence with at least 95% identity to any one of SEQ ID NOs: 1, 2, and 4, and the RNA guide comprises SEQ ID NO: 5 or SEQ ID NO: 6;
(c) the nuclease comprises any one of SEQ ID NOs: 1, 2, and 4, and the RNA guide comprises a nucleotide sequence with at least 95% sequence identity to SEQ ID NO: 5 or SEQ ID NO: 6;
(d) the nuclease comprises any one of SEQ ID NOs: 1, 2, and 4, and the RNA guide comprises SEQ ID NO: 5 or SEQ ID NO: 6;
(e) the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8;
(f) the nuclease comprises an amino acid sequence with at least 95% identity to SEQ ID NO: 3, and the RNA guide comprises SEQ ID NO: 7 or SEQ ID NO: 8;
(g) the nuclease comprises a SEQ ID NO: 3, and the RNA guide comprises a nucleotide sequence with at least 95% sequence identity to SEQ ID NO: 7 or SEQ ID NO: 8; or
(h) the nuclease comprises SEQ ID NO: 3, and the RNA guide comprises SEQ ID NO: 7 or SEQ ID NO: 8.

Embodiment 20 provides the composition of any of embodiments 16-19, wherein the composition is a delivery composition comprising a nanoparticle, a liposome, an exosome, a microvesicle, or a gene-gun.

Embodiment 21 provides a cell comprising the nuclease or the nucleic acid encoding the nuclease, the RNA guide or the nucleic acid encoding the RNA guide, or the composition of any previous embodiment.

Embodiment 22 provides a method of modifying a target nucleic acid in a cell, comprising delivering to the cell:
(a) a nuclease or a nucleic acid encoding the nuclease, wherein the nuclease comprises an amino acid sequence with at least 80% identity to any one of SEQ ID NOs: 1-4; and
(b) an RNA guide or a nucleic acid encoding the RNA guide, wherein the RNA guide comprises a spacer sequence and a direct repeat sequence and the spacer sequence recognizes the target nucleic acid.

Embodiment 23 provides the method of embodiment 22, wherein the nuclease comprises a RuvC domain or a split RuvC domain.

Embodiment 24 provides the method of embodiment 23, wherein the nuclease comprises a catalytic residue (e.g., aspartic acid or glutamic acid).

Embodiment 25 provides the method of any one of embodiments 22-24, wherein the nuclease comprises one or more of the following sequences:
(a) LX₁RLX₂LX₃GYEGRDDGX₄YEE (SEQ ID NO: 11), wherein X₁ is L or I, X₂ is F or I, X₃ is S or A, and X₄ is F or I;
(b) TREAYAX₁QQ (SEQ ID NO: 12), wherein X₁ is E or D;
(c) LX₁RYX₂ENGX₃RPR (SEQ ID NO: 13), wherein X₁ is F or L, X₂ is D or E, and X₃ is K or L;
(d) KEPRX₁CWRR (SEQ ID NO: 14), wherein X₁ is F or L;
(e) GX₁HX₂AFX₃RX₄WPKEVEGLD (SEQ ID NO: 15), wherein X₁ is K or D, X₂ is S or L, X₃ is T or S, and X₄ is Q or Y;
(f) DPX₁NX₂HNQIKEX₃DV (SEQ ID NO: 16), wherein X₁ is G or K, X₂ is K or E, and X₃ is K or S;
(g) SLKKX₁LHFGGYE (SEQ ID NO: 17), wherein X₁ is C or T; and
(h) ALTRX₁AEPF (SEQ ID NO: 18), wherein X₁ is E or H.

Embodiment 26 provides the method of any one of embodiments 22-25, wherein the nuclease comprises an amino acid sequence with at least 95% identity to any one of SEQ ID NOs: 1-4.

Embodiment 27 provides the method of any one of embodiments 22-26, wherein the nuclease comprises any one of SEQ ID NOs: 1-4.

Embodiment 28 provides the method of any one of embodiments 22-27, wherein the RNA guide comprises a nucleotide sequence with at least 95% sequence identity to any one of SEQ ID NOs: 5-8.

Embodiment 29 provides the method of embodiment 28, wherein the RNA guide comprises any one of SEQ ID NOs: 5-8.

Embodiment 30 provides the method of any one of embodiments 22-29, wherein:
(a) the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6;
(b) the nuclease comprises an amino acid sequence with at least 95% identity to any one of SEQ ID NOs: 1, 2, and 4, and the RNA guide comprises SEQ ID NO: 5 or SEQ ID NO: 6;
(c) the nuclease comprises any one of SEQ ID NOs: 1, 2, and 4, and the RNA guide comprises a nucleotide sequence with at least 95% sequence identity to SEQ ID NO: 5 or SEQ ID NO: 6;
(d) the nuclease comprises any one of SEQ ID NOs: 1, 2, and 4, and the RNA guide comprises SEQ ID NO: 5 or SEQ ID NO: 6;
(e) the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8;
(f) the nuclease comprises an amino acid sequence with at least 95% identity to SEQ ID NO: 3, and the RNA guide comprises SEQ ID NO: 7 or SEQ ID NO: 8;
(g) the nuclease comprises a SEQ ID NO: 3, and RNA guide comprises a nucleotide sequence with at least 95% sequence identity to SEQ ID NO: 7 or SEQ ID NO: 8; or
(h) the nuclease comprises SEQ ID NO: 3, and the RNA guide comprises SEQ ID NO: 7 or SEQ ID NO: 8.

Embodiment 31 provides the method of any one of embodiments 22-30, wherein the RNA guide comprises a spacer sequence between 10 and 50 nucleotides in length.

Embodiment 32 provides the method of any one of embodiments 22-30, wherein the RNA guide comprises a spacer sequence between 15 and 25 nucleotides in length.

Embodiment 33 provides the method of embodiment 31 or 32, wherein the spacer sequence comprises about 20 nucleotides in length.

Embodiment 34 provides the method of one of embodiments 22-33, wherein the RNA guide recognizes the target nucleic acid comprising a target sequence adjacent to a protospacer adjacent motif (PAM) sequence, wherein the PAM sequence comprises a nucleotide sequence set forth as 5′-TTN-3′ or 5′-NTTN-3′, wherein “N” is any nucleobase.

Embodiment 35 provides the method of embodiment 34, wherein the PAM sequence comprises a nucleotide sequence set forth as 5′-TTA-3′, 5′-TTT-3′, 5′-TTG-3′, 5′-TTC-3′, 5′-NTTA-3′, 5′-NTTT-3′, 5′-NTTG-3′, or 5′-NTTC-3′.

Embodiment 36 provides the method of embodiment 35, wherein the PAM sequence comprises a nucleotide sequence set forth as 5′-GTTG-3′.

Embodiment 37 provides the method of any one of embodiments 22-36, wherein the nuclease introduces a single-stranded break or a double-stranded break into the target nucleic acid.

Embodiment 38 provides the method of any one of embodiments 22-37, wherein the modification is an insertion, deletion, and/or substitution into the target nucleic acid.

Embodiment 39 provides a method of binding a nuclease and an RNA guide to a target nucleic acid in a cell, the method comprising:
(a) delivering the nuclease or the nucleic acid encoding the nuclease of any one of embodiments 1-6, and the RNA guide or the nucleic acid encoding the RNA guide of any one of embodiments 7-15 to the cell; or
(b) delivering the composition of any one of embodiments 16-19 to the cell.

Embodiment 40 provides a method of introducing an insertion, deletion, or substitution into a target nucleic acid in a cell, the method comprising:
(a) delivering the nuclease or the nucleic acid encoding the nuclease of any one of embodiments 1-6, and the RNA guide or the nucleic acid encoding the RNA guide of any one of embodiments 7-15 to the cell; or
(b) delivering the composition of any one of embodiments 16-19 to the cell.

Embodiment 41 provides the method of any one of embodiments 22-40, wherein the cell is a eukaryotic cell.

Embodiment 42 provides the method of any one of embodiments 22-40, wherein the cell is a prokaryotic cell.

Embodiment 43 provides the method of any one of embodiments 22-40, wherein the cell is a human cell.

Embodiment 44 provides the nuclease or the nucleic acid encoding the nuclease, the composition, or the method of any previous embodiments, wherein the nucleic acid encoding the nuclease is operably linked to a promoter.

Embodiment 45 provides the nuclease or the nucleic acid encoding the nuclease, the composition, or the method of any previous embodiment, wherein the nucleic acid encoding the nuclease is in a vector.

Embodiment 46 provides the nuclease or the nucleic acid encoding the nuclease, the composition, or the method of embodiment 45, wherein the vector comprises a retroviral vector, a lentiviral vector, a phage vector, an adenoviral vector, an adeno-associated vector, or a herpes simplex vector.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Analysis of Amino Acid Sequences

In this Example, amino acid sequences of the sequences of SEQ ID NOs: 1-4 were analyzed to identify potential functional protein domains. The amino acid sequences were determined to include a putative C-terminal RuvC domain.

The amino acid sequences of SEQ ID NOs: 1-4 were further aligned to identify regions of sequence similarity, as shown in FIG. 1A and FIG. 1B. The consensus sequence is set forth at the top of FIG. 1A and FIG. 1B. Below the consensus sequence, a bar graph depicts sequence similarity, with the tallest bars indicating the residues with the highest sequence similarity. Non-limiting regions of sequence similarity are shown in Table 3.

TABLE 3
Conserved Sequences.

| Sequence | Residues |
|---|---|
| LX₁RLX₂LX₃GYEGRDDGX₄YEE | X₁ is L or I |
| (SEQ ID NO: 11) | X₂ is F or I |
| | X₃ is S or A |
| | X₄ is F or I |
| TREAYAX₁QQ (SEQ ID NO: 12) | X₁ is E or D |
| LX₁RYX₂ENGX₃RPR (SEQ ID NO: 13) | X₁ is F or L |
| | X₂ is D or E |
| | X₃ is K or L |
| KEPRX₁CWRR (SEQ ID NO: 14) | X₁ is F or L |
| GX₁HX₂AFX₃RX₄WPKEVEGLD | X₁ is K or D |
| (SEQ ID NO: 15) | X₂ is S or L |
| | X₃ is T or S |
| | X₄ is Q or Y |
| DPX₁NX₂HNQIKEX₃DV (SEQ ID NO: 16) | X₁ is G or K |
| | X₂ is K or E |
| | X₃ is K or S |
| SLKKX₁LHFGGYE (SEQ ID NO: 17) | X₁ is C or T |
| ALTRX₁AEPF (SEQ ID NO: 18) | X₁ is E or H |

This Example indicates that the amino acid sequences of SEQ ID NOs: 1-4 were classified as a family with a conserved RuvC domain representative of nucleases.

### Example 2—Expression of Nucleases in E. coli

In this Example, a system individually comprising a nuclease of any one of SEQ ID NOs: 2-4 was engineered and introduced into E. coli.

For each nuclease, a polynucleotide encoding the nuclease was E. coli codon-optimized, synthesized (Genscript®), and individually cloned into a custom expression system derived from pET-28a(+) (EMD-Millipore). The vector included a polynucleotide encoding each nuclease under the control of a lac promoter and an E. coli ribosome binding sequence. The vector also included a site for a pre-crRNA (direct repeat-spacer-direct repeat) driven by a J23119 promoter following the open reading frame for the nuclease. For each nuclease, the direct repeat sequences tested are set forth in Table 2. The spacers were designed to target sequences of a pACYC184 plasmid and E. coli essential genes.

The nuclease/pre-crRNA plasmids were electroporated into E. Cloni® 10G electrocompetent E. coli (Lucigen®). The nuclease/pre-crRNA plasmids were either co-transformed with purified pACYC184 plasmid or directly transformed into pACYC184-containing E. Cloni® 10G electrocompetent E. coli (Lucigen®), plated onto agar containing the proper antibiotics, and incubated for 10-12 hours at 37° C.

A proxy for activity of the engineered nuclease/pre-crRNA system in E. coli was investigated, wherein bacterial cell death was used as the proxy for system activity. An active nuclease associated with a pre-crRNA could disrupt expression of a spacer sequence target, e.g., a pACYC184 plasmid sequence or an E. coli essential gene, resulting in cell death. Using this proxy, the nucleases disclosed herein were determined to have activity in E. coli.

Thus, this Example suggests that the nucleases of SEQ ID NOs: 2-4 were capable of being expressed in bacterial cells. With a pre-crRNA (direct repeat-spacer-direct repeat), the nucleases of SEQ ID NOs: 2-4 were shown to have activity in bacterial cells.

### Example 3—Targeting of Mammalian Gene by Nuclease of SEQ ID NO: 4

This Example describes an indel assessment on a mammalian VEGFA target by the nuclease of SEQ ID NO: 4 introduced into mammalian cells by transient transfection.

The nuclease of SEQ ID NO: 4 was cloned into a pcda3.1 backbone (Invitrogen™). The plasmid was then maxi-prepped and diluted to 1 μg/μL. For RNA guide preparation, a dsDNA fragment encoding an RNA guide was derived by ultramers containing the target sequence scaffold, and the U6 promoter. Ultramers™ were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the RNA guide was done in 50 μL reactions with the following components: 0.02 μl of aforementioned template, 2.5 μl forward primer, 2.5 μl reverse primer, 25 μL HiFi Q5® Polymerase (New England Biolabs®), and 20 μl water. Cycling conditions were: 1×(30 s at 98° C.), 30×(10 s at 98° C., 15 s at 67° C.), 1×(2 min at 72° C.). PCR products were cleaned up with a 1.8×SPRI treatment and normalized to 25 ng/μL. The sequence of the VEGFA target locus tested was GGTAAAGGTATTGGGAGGTTAGAGT (SEQ ID NO: 9), and the corresponding crRNA sequence was GUGUAGGCCUCCUCUGAAUGGGGUGGCUAAUGACACGGUAAAGGUAUUGGGAGGUUA GAGU (SEQ ID NO: 10). The target of SEQ ID NO: 9 was adjacent to a 5′-GTTG-3′ PAM sequence.

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine™ 2000 and 9.5 μl of Opti-MEM® was prepared and then incubated at room temperature for 5-20 minutes (Solution 1). After incubation, the lipofectamine:OptiMEM mixture was added to a separate mixture containing 182 ng of nuclease plasmid and 14 ng of crRNA and water up to 10 μL (Solution 2). In the case of negative controls, the crRNA was not included in Solution 2. The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE™ to the center of each well and incubated for approximately 5 minutes. 100 μL of D10 media was then added to each well and mixed to resuspend cells. The cells were then spun down at 500 g for 10 minutes, and the supernatant was discarded. QuickExtract™ buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

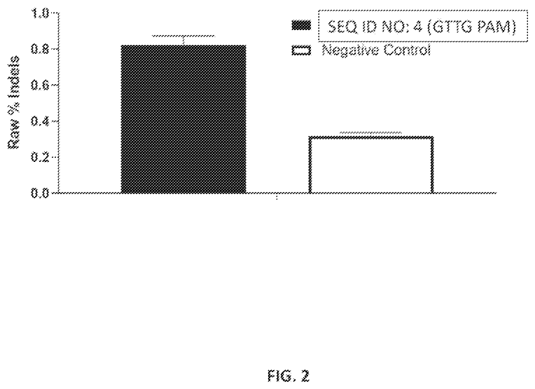

FIG. 2 shows percent indels in the VEGFA target locus in HEK293T cells following transfection with the nuclease of SEQ ID NO: 2. The dots reflect percent indels measured in two bioreplicates, and the bars reflect the mean percent indels measured in the two bioreplicates. The closed dots represent indels induced by the nuclease of SEQ ID NO: 4, and the open dots represent indels measured in the negative control samples. For the nuclease of SEQ ID NO: 4, the percent indels were higher than the percent indels of the negative control.

This Example suggests that the nuclease of SEQ ID NO: 4 has nuclease activity in mammalian cells.

1. A nuclease or a nucleic acid encoding the nuclease, wherein the nuclease comprises an amino acid sequence with at least 80% identity to any one of SEQ ID NOs: 1-4.

2.-3. (canceled)

4. The nuclease or the nucleic acid encoding the nuclease of claim 1, wherein the nuclease comprises one or more of the following sequences:
(a) LXiRLX₂LX₃GYEGRDDGX₄YEE (SEQ ID NO: 11), wherein X₁ is L or I, X₂ is F or I, X₃ is S or A, and X₄ is F or I;
(b) TREAYAX₁QQ (SEQ ID NO: 12), wherein X₁ is E or D;
(c) LX₁RYX₂ENGX₃RPR (SEQ ID NO: 13), wherein X₁ is F or L, X₂ is D or E, and X₃ is K or L;
(d) KEPRX₁CWRR (SEQ ID NO: 14), wherein X₁ is F or L;
(e) GX₁HX₂AFX₃RX₄WPKEVEGLD (SEQ ID NO: 15), wherein X₁ is K or D, X₂ is S or L, X₃ is T or S, and X₄ is Q or Y;
(f) DPX₁NX₂HNQIKEX₃DV (SEQ ID NO: 16), wherein X₁ is G or K, X₂ is K or E, and X₃ is K or S;
(g) SLKKX₁LHFGGYE (SEQ ID NO: 17), wherein X₁ is C or T; and
(h) ALTRX₁AEPF (SEQ ID NO: 18), wherein X₁ is E or H.

5. The nuclease or the nucleic acid encoding the nuclease of claim 1, wherein the nuclease comprises an amino acid sequence with at least 95% identity to any one of SEQ ID NOs: 1-4.

6. The nuclease or the nucleic acid encoding the nuclease of claim 1, wherein the nuclease comprises any one of SEQ ID NOs: 1-4.

7. An RNA guide or a nucleic acid encoding the RNA guide, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

8. The RNA guide or the nucleic acid encoding the RNA guide of claim 7, wherein the direct repeat sequence comprises any one of SEQ ID NOs: 5-8.

9.-15. (canceled)

16. A composition comprising the nuclease or the nucleic acid encoding the nuclease of claim 1.

17. A composition comprising the RNA guide or the nucleic acid encoding the RNA guide of claim 7.

18. A composition comprising: a) the nuclease or the nucleic acid encoding the nuclease of claim 1, and b) an RNA guide or a nucleic acid encoding the RNA guide, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

19. The composition of claim 18, wherein:
(a) the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6;
(b) the nuclease comprises an amino acid sequence with at least 95% identity to any one of SEQ ID NOs: 1, 2, and 4, and the RNA guide comprises SEQ ID NO: 5 or SEQ ID NO: 6;
(c) the nuclease comprises any one of SEQ ID NOs: 1, 2, and 4, and the RNA guide comprises a nucleotide sequence with at least 95% sequence identity to SEQ ID NO: 5 or SEQ ID NO: 6;
(d) the nuclease comprises any one of SEQ ID NOs: 1, 2, and 4, and the RNA guide comprises SEQ ID NO: 5 or SEQ ID NO: 6;
(e) the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8;
(f) the nuclease comprises an amino acid sequence with at least 95% identity to SEQ ID NO: 3, and the RNA guide comprises SEQ ID NO: 7 or SEQ ID NO: 8;
(g) the nuclease comprises SEQ ID NO: 3, and the RNA guide comprises a nucleotide sequence with at least 95% sequence identity to SEQ ID NO: 7 or SEQ ID NO: 8; or
(h) the nuclease comprises SEQ ID NO: 3, and the RNA guide comprises SEQ ID NO: 7 or SEQ ID NO: 8.

20. (canceled)

21. A cell comprising the nuclease or the nucleic acid encoding the nuclease of claim 1, and optionally, an RNA guide or a nucleic acid encoding the RNA guide, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

22. A method of modifying a target nucleic acid in a cell, the method comprising delivering to the cell:
(a) a nuclease or a nucleic acid encoding the nuclease, wherein the nuclease comprises an amino acid sequence with at least 80% identity to any one of SEQ ID NOs: 1-4; and
(b) an RNA guide or a nucleic acid encoding the RNA guide, wherein the RNA guide comprises a spacer sequence and a direct repeat sequence, and further wherein the spacer sequence recognizes the target nucleic acid.

23.-24. (canceled)

25. The method of claim 22, wherein the nuclease comprises one or more of the following sequences:
(a) LX₁RLX₂LX₃GYEGRDDGX₄YEE (SEQ ID NO: 11), wherein X₁ is L or I, X₂ is F or I, X₃ is S or A, and X₄ is F or I;
(b) TREAYAX₁QQ (SEQ ID NO: 12), wherein X₁ is E or D;
(c) LX₁RYX₂ENGX₃RPR (SEQ ID NO: 13), wherein X₁ is F or L, X₂ is D or E, and X₃ is K or L;
(d) KEPRX₁CWRR (SEQ ID NO: 14), wherein X₁ is F or L;
(e) GX₁HX₂AFX₃RX₄WPKEVEGLD (SEQ ID NO: 15), wherein X₁ is K or D, X₂ is S or L, X₃ is T or S, and X₄ is Q or Y;
(f) DPX₁NX₂HNQIKEX₃DV (SEQ ID NO: 16), wherein X₁ is G or K, X₂ is K or E, and X₃ is K or S;
(g) SLKKX₁LHFGGYE (SEQ ID NO: 17), wherein X₁ is C or T; and
(h) ALTRX₁AEPF (SEQ ID NO: 18), wherein X₁ is E or H.

26. The method of claim 22, wherein the nuclease comprises an amino acid sequence with at least 95% identity to any one of SEQ ID NOs: 1-4.

27. The method of claim 22, wherein the nuclease comprises any one of SEQ ID NOs: 1-4.

28. The method of claim 22, wherein the RNA guide comprises a nucleotide sequence with at least 95% sequence identity to any one of SEQ ID NOs: 5-8.

29. The method of claim 28, wherein the RNA guide comprises any one of SEQ ID NOs: 5-8.

30. The method of claim 22, wherein:
(a) the nuclease comprises an amino acid sequence with at least 95% identity to any one of SEQ ID NOs: 1, 2, and 4, and the RNA guide comprises a nucleotide sequence with at least 95% sequence identity to SEQ ID NO: 5 or SEQ ID NO: 6;
(b) the nuclease comprises an amino acid sequence with at least 95% identity to any one of SEQ ID NOs: 1, 2, and 4, and the RNA guide comprises SEQ ID NO: 5 or SEQ ID NO: 6;
(c) the nuclease comprises any one of SEQ ID NOs: 1, 2, and 4, and the RNA guide comprises a nucleotide sequence with at least 95% sequence identity to SEQ ID NO: 5 or SEQ ID NO: 6;
(d) the nuclease comprises any one of SEQ ID NOs: 1, 2, and 4, and the RNA guide comprises SEQ ID NO: 5 or SEQ ID NO: 6;
(e) the nuclease comprises an amino acid sequence with at least 95% identity to SEQ ID NO: 3, and the RNA guide comprises a nucleotide sequence with at least 95% sequence identity to SEQ ID NO: 7 or SEQ ID NO: 8;
(f) the nuclease comprises an amino acid sequence with at least 95% identity to SEQ ID NO: 3, and the RNA guide comprises SEQ ID NO: 7 or SEQ ID NO: 8;
(g) the nuclease comprises SEQ ID NO: 3, and RNA guide comprises a nucleotide sequence with at least 95% sequence identity to SEQ ID NO: 7 or SEQ ID NO: 8; or
(h) the nuclease comprises SEQ ID NO: 3, and the RNA guide comprises SEQ ID NO: 7 or SEQ ID NO: 8.

31.-38. (canceled)

39. A method of binding a nuclease and an RNA guide to a target nucleic acid in a cell, the method comprising
delivering the nuclease or the nucleic acid encoding the nuclease of claim 1, and an RNA guide or a nucleic acid encoding the RNA guide to the cell, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

40. A method of introducing an insertion, deletion, or substitution into a target nucleic acid in a cell, the method comprising
delivering the nuclease or the nucleic acid encoding the nuclease of claim 1, and an RNA guide or a nucleic acid encoding the RNA guide to the cell, wherein the RNA guide or the nucleic acid encoding the RNA guide comprises (i) a direct repeat sequence comprising a nucleotide sequence with at least 95% sequence identity to any one of SEQ ID NOs: 5-8, and (ii) a spacer sequence.

41.-46. (canceled)


SEQUENCE LISTING (20 sequences)

SEQ ID NO: 1 - protein, 821 aa, Artificial Sequence
  Nuclease
MAFQSKRRIVGNFVKEQCLKAVDGKVILTDQEKRELIKRYELHLEPHKWLLRLFLSGYEGRDDGFYEELGNTNLDKEKFFEVTAGLRDALLRQSGSSRALKSSMLGKCPPSAAVGKAAKHIQTLRDAGILPFKTGLTSGEDYNVLQQAVQQLRSWVACDHRTREAYAEQQEKTSQAEEAAKKAANEVKPEDAKSLERHERVLTKLRKQERRLERMKSHAQFSLDEMDCTGYSLCMGANYLKDYCLEKEGRGLRLTLKNSTMAGSYYVSVGDGQHAGMKNPGTPAGGSPEKGRRRNILFDFTVEKCGDNYLFRYDENGKRPRAGVVKEPRFCWRRKGNSVELYLAMPINIENSMRNIFVGKQKSGKHSAFTRQWPKEVEGLDELRDAVVLGVDIGINRAAFCAALKTSRFENGLPADVQVMDTTCDALTEKGQEYRQLRKDATCLAWLIRTTRRFKADPGNKHNQIKEKDVERFDSADGAYRRYMDAIAEMPSDPLQVWEAARITGYGEWAKEIFARFNHYKHEHACCAVSLSLSDRLVWCRLIDRILSLKKCLHFGGYESKHRKGFCKSLYRLRHNARNDVRKKLARFIVDAAVDAGASVIAMEKLPSSGGKQSKDDNRIWDLMAPNTLATTVCLMAKVEGIGFVQVDPEFTSQWVFEQRVIGDREGRIVSCLDAEGVRRDYDADENAAKNIAWLALTREAEPFCMAFEKRNGVVEPKGLRFDIPEEPTREQDESDQDFKKRLEERDKLIERLQAKADRMQAIVQRLFGDRRPWDAFADRIPEGKSKRLFRHRDGLVLNKPFKGLCGSENSEQKASARNSR

SEQ ID NO: 2 - protein, 821 aa, Artificial Sequence
  Nuclease
MAFQSKRRIVGNFVKEQCLKAVDGKVILTDQEKRELIKRYELHLEPHKWLLRLFLSGYEGRDDGFYEELGNTNLDKEKFFEVTAGLRDALLRQSGSSRALKSSMLGKCPPSAAVGKAAKHIQTLRDAGILPFKTGLTSGEDYNVLQQAVQQLRSWVACDHRTREAYAEQQEKTSQAEEAAKKAANEVKPEDAKSLERHERVLTKLRKQERRLERMKSHAQFSLDEMDCTGYSLCMGANYLKDYCLEKEGRGLRLTLKNSTMAGSYYVSVGDGQHAGMKNPGTPAGGSPEKGRRRNILFDFTVEKCGDNYLFRYDENGKRPRAGVVKEPRFCWRRKGNSVELYLAMPINIENSMRNIFVGKQKSGKHSAFTRQWPKEVEGLDELRDAVVLGVDIGINRAAFCAALKTSRFENGLPADVQVMDTTCDALTEKGQEYRQLRKDATCLAWLIRTTRRFKADPGNKHNQIKEKDVERFDSADGAYRRYMDAIAEMPSDPLQVWEAARITGYGEWAKEIFARFNHYKHEHACCAVSLSLSDRLVWCRLIDRILSLKKCLHFGGYESKHRKGFCKSLYRLRHNARNDVRKKLARFIVDAAVDAGASVIAMEKLPSSGGKQSKDDNRIWDLMAPNTLATTVCLMAKVEGIGFVQVDPEFTSQWVFEQRVIGDREGRIVSCLDAEGVRRDYDADENAAKNIAWLALTREAEPFCMAFEKRNGVVEPKGLRFDIPEEPTREQDESDQDFKKRLEERDKLIERLQAKADRMQAIVQRLFGDRRPWDAFADRIPEGKSKRLFRHRDGLVLNKPFKGLCGSENSGQKASARNSR

SEQ ID NO: 3 - protein, 837 aa, Artificial Sequence
  Nuclease
MGRFGKKKIAVNGYVEQDCIKTISAKCLLTRAQIDELRAKYDAVLDTMRPLIRLILAGYEGRDDGIYEEIAPEMSKKKFFEAATEWRESIVKNASPRAMKASVFGDKEPCKSTGGARAVIGKLRKSGVFPIETGLSGGDEYNLIEQAIEYAKSWLKSDEATREAYADQQKDIKRLIGEAKKLALKIEKAEKKLEATNPQTKSWKKTTEIIKKSKREFGSVTTKTEKAEKRFERMKPFSKLELQNMDCTKYSTYLGTNYSPFKLKKEGDLLQITVTSSVMKGTYLASYGDGQYGSRRNNGQSRRDDFVPNMNQKRRRNLMFDCTVEPFGDGSLLRYEENGLRPRVAELKEPRLCWRRRNGNYELYLMMPVKMHVKSPEMFAGDHLAFSRYWPKEVEGLDSDTKITALGVDVGIIRSAYCVAVTAERFVDGLPTEMTVGKASFDAQTEKGREYFELGRRATMLGWLIKTTRRYKKDPKNEHNQIKESDVAAFDGSPGAFEHYILAVDEMSDDPLDVWGHANITGYGKWTKQIFKEFNQLKRERAEGQVEPNMTDDLTWCSLIDYIISLKKTLHFGGYETKERESFCPALYNERANCRDVVRKRLARYVVERAIAAEAQVISVENLSKCRRDDKRKNRVWDLMSQQSWIGVLTNMARMENIAVVSVNPDLTSQWVEQCGAIGDRKARTIACRDVNGKFVSLDADLNAAYNIASRALTRHAEPFSITFKKKDGILEQKDVCFDPGVIPVLEKNENEEKFRERVEKYEKSLVIKQERAVRWRAILQHLFGNERPWDEFTDEVKEGRHVSLYRHHGKLVRTKQYAGLVKEANNELVPVCAVAR

SEQ ID NO: 4 - protein, 821 aa, Artificial Sequence
  Nuclease
MAFQSKRRIVGNLVKEQCLKAVDGKVILTDQEKRELIKRYELHLEPYKWLLRLFLSGYEGRDDGFYEELGNTNLDKEKFFEVTAGLRDALLRQSGSSRALKSSMLGKCPPSAAVGKAAKHIQALRDAGILPFKTGLTSGEDYNVLQQAVQQLRSWVACDHRTREAYAEQQEKTSQAEEAAKKAVNEVKPEDAKSLERHERALTKLRKQERRLERMRSHAQFSLDEMDCTGYSLCMGANYLKDYCLEKEGRGLRLTLKNSTMAGSYYVSVGDGQHAGMKNPGTPAGGSPEKGRRRNILFDFAVEKCGDNYLFRYDENGKRPRAGVVKEPRFCWRRKGNSVELYLAMPINIENSMRNIFVGKQKSGKHSAFTRQWPKEVEGLDELRDAVVLGVDIGINRAAFCAALKTSRFENGLPADVQVMDTTCDALTEKGQEYRQLRKDATCLAWLIRTTRRFKADPGNKHNQIKEKDVERFDSADGAYRRYMDAIAEMPSDPLQVWEAARITGYGEWAKEIFARFNHYKHEHACCTVSLSLSDRLVWCRLIDRILSLKKCLHFGGYESKHRKGFCKSLYRLRHNARNDVRKKLARFVVDAAVDAGASVIAMEKLPSSGGKQSRDDNRIWDLMAPNTLATTVCLMAKVEGIGFVQVDPEFTSQWVFEQRVIGDREGRIVSCLDAEGVRRDYDADENAAKNIAWLALTREAEPFCMAFEKRNGVVEPKGFRFDIPEEPTREQDESNQDFKKRLEERDKLIERLQAKSDRMRAIVRRLFGDRRPWDAFADRIPEGKSKRLFRHRDGLVLNKPFKGLCGSENSEQKASARNSR

SEQ ID NO: 5 - RNA, 36 nt, Artificial Sequence
  Direct Repeat
guguaggccuccucugaaugggguggcuaaugacac

SEQ ID NO: 6 - RNA, 36 nt, Artificial Sequence
  Direct Repeat
gugucauuagccaccccauucagaggaggccuacac

SEQ ID NO: 7 - RNA, 20 nt, Artificial Sequence
  Direct Repeat
cauucagaacggaucaacac

SEQ ID NO: 8 - RNA, 20 nt, Artificial Sequence
  Direct Repeat
guguugauccguucugaaug

SEQ ID NO: 9 - DNA, 25 bp, Artificial Sequence
  VEGFA target locus
ggtaaaggtattgggaggttagagt

SEQ ID NO: 10 - RNA, 61 nt, Artificial Sequence
  crRNA
guguaggccuccucugaaugggguggcuaaugacacgguaaagguauugggagguuagagu

SEQ ID NO: 11 - protein, 19 aa, Artificial Sequence
  Nuclease
  MISC_FEATURE (2)..(2): Xaa is L or I
  MISC_FEATURE (5)..(5): Xaa is F or I
  MISC_FEATURE (7)..(7): Xaa is S or A
  MISC_FEATURE (16)..(16): Xaa is F or I
LXRLXLXGYEGRDDGXYEE

SEQ ID NO: 12 - protein, 9 aa, Artificial Sequence
  Nuclease
  MISC_FEATURE (7)..(7): Xaa is E or D
TREAYAXQQ

SEQ ID NO: 13 - protein, 12 aa, Artificial Sequence
  Nuclease
  MISC_FEATURE (2)..(2): Xaa is F or L
  MISC_FEATURE (5)..(5): Xaa is D or E
  MISC_FEATURE (9)..(9): Xaa is K or L
LXRYXENGXRPR

SEQ ID NO: 14 - protein, 9 aa, Artificial Sequence
  Nuclease
  MISC_FEATURE: Xaa is F or L
  misc_feature (5)..(5): Xaa can be any naturally occurring amino acid
KEPRXCWRR

SEQ ID NO: 15 - protein, 18 aa, Artificial Sequence
  Nuclease
  MISC_FEATURE (2)..(2): Xaa is K or D
  MISC_FEATURE (4)..(4): Xaa is S or L
  MISC_FEATURE (7)..(7): Xaa is T or S
  MISC_FEATURE (9)..(9): Xaa is Q or Y
GXHXAFXRXWPKEVEGLD

SEQ ID NO: 16 - protein, 14 aa, Artificial Sequence
  Nuclease
  MISC_FEATURE (3)..(3): Xaa is G or K
  MISC_FEATURE (5)..(5): Xaa is K or E
  MISC_FEATURE (12)..(12): Xaa is K or S
DPXNXHNQIKEXDV

SEQ ID NO: 17 - protein, 12 aa, Artificial Sequence
  Nuclease
  MISC_FEATURE (5)..(5): Xaa is C or T
SLKKXLHFGGYE

SEQ ID NO: 18 - protein, 9 aa, Artificial Sequence
  Nuclease
  MISC_FEATURE (5)..(5): Xaa is E or H
ALTRXAEPF

SEQ ID NO: 19 - protein, 846 aa, Artificial Sequence
  FIG. 1A Consensus Sequence
  MISC_FEATURE (3)..(3): Xaa is space
  MISC_FEATURE (14)..(14): Xaa is F, Y, or L
  MISC_FEATURE (48)..(48): Xaa is H, M, or Y
  MISC_FEATURE (124)..(124): Xaa is T, K, or A
  MISC_FEATURE (196)..(211): Xaa is space
  MISC_FEATURE (218)..(218): Xaa is space, E, or A
  MISC_FEATURE (219)..(221): Xaa is space
  MISC_FEATURE (308)..(311): Xaa is space
  MISC_FEATURE (553)..(553): Xaa is A, Q, or T
  MISC_FEATURE (614)..(614): Xaa is I or V
  MISC_FEATURE (745)..(745): Xaa is L, V, or F
  MISC_FEATURE (761)..(761): Xaa is D, E, or N
  MISC_FEATURE (786)..(786): Xaa is Q or R
MAXFQSKRRIVGNXVKEQCLKAVDGKVILTDQEKRELIKRYELHLEPXKWLLRLFLSGYEGRDDGFYEELGNTNLDKEKFFEVTAGLRDALLRQSGSSRALKSSMLGKCPPSAAVGKAAKHIQXLRDAGILPFKTGLTSGEDYNVLQQAVQQLRSWVACDHRTREAYAEQQEKTSQAEEAAKKAANEVKPEDAKSXXXXXXXXXXXXXXXXLERHERXXXXVLTKLRKQERRLERMKSHAQFSLDEMDCTGYSLCMGANYLKDYCLEKEGRGLRLTLKNSTMAGSYYVSVGDGQHAGMKNPGTPAGGXXXXSPEKGRRRNILFDFTVEKCGDNYLFRYDENGKRPRAGVVKEPRFCWRRKGNSVELYLAMPINIENSMRNIFVGKQKSGKHSAFTRQWPKEVEGLDELRDAVVLGVDIGINRAAFCAALKTSRFENGLPADVQVMDTTCDALTEKGQEYRQLRKDATCLAWLIRTTRRFKADPGNKHNQIKEKDVERFDSADGAYRRYMDAIAEMPSDPLQVWEAARITGYGEWAKEIFARFNHYKHEHACCXVSLSLSDRLVWCRLIDRILSLKKCLHFGGYESKHRKGFCKSLYRLRHNARNDVRKKLARFXVDAAVDAGASVIAMEKLPSSGGKQSKDDNRIWDLMAPNTLATTVCLMAKVEGIGFVQVDPEFTSQWVFEQRVIGDREGRIVSCLDAEGVRRDYDADENAAKNIAWLALTREAEPFCMAFEKRNGVVEPKGXRFDIPEEPTREQDESXQDFKKRLEERDKLIERLQAKADRMXAIVQRLFGDRRPWDAFADRIPEGKSKRLFRHRDGLVLNKPFKGLCGSENSEQKASARNSR

SEQ ID NO: 20 - protein, 821 aa, Artificial Sequence; identical to SEQ ID NO: 1
  FIG. 1B Consensus Sequence